(19) United States
(12) Patent Application Publication
(10) Pub. No.: US 2021/0267986 A1
(43) Pub. Date: Sep. 2, 2021

(54) THERAPEUTIC REGIMENS FOR TREATMENT OF CANCER USING ERIBULIN AND SELECTIVE CDK4/6 INHIBITOR COMBINATIONS

(71) Applicant: G1 Therapeutics, Inc., Research Triangle Park, NC (US)

(72) Inventors: Jessica A. Sorrentino, Durham, NC (US); Patrick Joseph Roberts, Durham, NC (US); Jay Copeland Strum, Hillsborough, NC (US)

(73) Assignee: G1 Therapeutics, Inc., Research Triangle Park, NC (US)

(21) Appl. No.: 17/315,011

(22) Filed: May 7, 2021

Related U.S. Application Data
(63) Continuation of application No. PCT/US2019/061010, filed on Nov. 12, 2019.
(60) Provisional application No. 62/758,388, filed on Nov. 9, 2018.

(51) Int. Cl. A61K 31/527; A61K 31/357; A61K 45/06; A61P 35/04

(52) U.S. Cl. CPC A61K 31/527; A61P 35/04; A61K 45/06; A61K 31/357

(57) ABSTRACT

The present invention provides methods and compositions for treating cancers with a combination of eribulin and a selective CDK4/6 inhibitor, wherein the selective CDK4/6 inhibitor reduces eribulin's effects on myelosuppression and/or myeloablation without reducing the efficacy of eribulin therapy.

### STATEMENT OF RELATED APPLICATIONS

This application is a continuation of International Patent Application No. PCT/US2019/061010, filed in the U.S. Receiving Office on Nov. 12, 2019, which claims priority to provisional U.S. Application No. 62/758,388, filed Nov. 9, 2018. The entirety of each these applications is hereby incorporated by reference herein for all purposes.

### FIELD OF THE INVENTION

The present invention provides therapeutic regimens and compositions for treating cancers with a combination of eribulin and a selective CDK4/6 inhibitor, wherein the selective CDK4/6 inhibitor reduces eribulin's effects on myelosuppression and/or myeloablation without reducing the efficacy of eribulin therapy.

### BACKGROUND OF THE INVENTION

Eribulin is an antineoplastic agent that is a synthetic derivative of the natural product halichondrin B, a compound isolated from sponges of the genus Halichondria. Eribulin has the chemical structure:

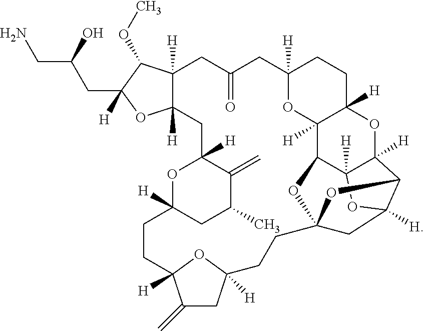

Eribulin is an inhibitor of microtubule dynamics that binds to the centromeric cap of the plus (+) ends of microtubules and suppresses microtubule growth by forming nonproductive tubulin aggregates. Eribulin does not affect microtubule shortening. The mechanism of action for eribulin differs from other clinically-important tubulin-targeting agents: vinca alkaloids such as vincristine and vinblastine bind to the plus ends and the sides of the microtubule, reducing the concentration of tubulin aggregates and microtubule shrinking; taxanes such as paclitaxel and docetaxel promote microtubule polymerization and maintain the microtubule structure, inhibiting the mitotic spindle; and epothilones such as ixabepilone induce microtubule polymerization through a mechanism similar to taxanes. In addition, eribulin exhibits effects beyond its cytotoxic, antimitotic mechanisms such as vascular remodeling that leads to increased tumor perfusion and reduced hypoxia, reversal of epithelial-mesenchymal transition, and decreased capacity for migration and invasion leading to reduced metastatic capacity. Eribulin is typically administered intravenously as its mesylate salt.

Eribulin mesylate has been approved by the United States Food and Drug Administration (FDA) for the treatment of patients with metastatic breast cancer who have received at least two prior chemotherapy regimens for late-stage disease including anthracycline- and taxane-based chemotherapies. Eribulin mesylate has also been approved by the U.S. FDA for the treatment of inoperable liposarcoma in patients who have received prior anthracycline-based chemotherapy. Eribulin is also currently being investigated for a variety of cancers and solid tumors including: non-small cell lung cancer; prostate cancer; relapsed/refractory rhabdomyosarcoma; non-rhabdomyosarcoma soft tissue sarcoma; Ewing sarcoma; angiosarcoma; epithelioid hemangioendothelioma; and metastatic urothelial cell cancer.

One major side effect of eribulin chemotherapy, however, can be severe hematologic toxicity of hematopoietic cells resulting in myelosuppression, and in some instances, myeloablation. Typically, patients receiving eribulin experiencing myelosuppression present with neutropenia. For example, patients receiving eribulin have an incidence of >grade 3 neutropenia around 57%. A patient that presents with an absolute neutrophil count (ANC) below 1,000/mm³ will often receive delayed dosing, a dosing “holiday,” or the cessation of treatment. Because eribulin is generally administered to patients with recurrent disease, these treatment delays may adversely affect the outcome of eribulin therapy.

WO 2019/017497 to Eisai R&D Management Co., LTD. describes methods for treating certain cancers by administering eribulin in combination with a cyclin dependent kinase 4/6 (CDK4/6) inhibitor, wherein the CDK4/6 inhibitor is explicitly withheld for a certain period of time during the treatment protocol prior to, during, or after administration of eribulin. For example, the CDK4/6 inhibitor is withheld for one or more days before, during, or after eribulin is administered, or the CDK4/6 inhibitor is withheld for two days before, during, or after eribulin is administered Thus, for example, the CDK4/6 inhibitor may not be administered within about 24-48 hours before eribulin, and/or the CDK4/6 inhibitor may not be administered within about 24 hours after eribulin. As described, the rationale for withholding the CDK4/6 inhibitor is that simultaneous exposure to the two drugs may result in cell cycle-based antagonism, in which the antimitotic activity of eribulin prevents cells from reaching the G1/S cell cycle checkpoint where a CDK4/6 inhibitor (e.g., palbociclib) exerts its CDK4/6 inhibitory activity, and the CDK4/6 inhibitory activity at the G1/S checkpoint prevents cells from reaching mitosis where eribulin exerts its antimitotic activity. To prevent such antagonism, a “CDK4/6 inhibitor holiday” is proposed. This “CDK4/6 holiday,” however, may fail to prevent the inherent toxicity associated with the use of eribulin.

Accordingly, there is a clear need for new eribulin chemotherapy regimens that reduce toxic side effects while still proving effective in treating the targeted cancer or tumor.

### SUMMARY OF THE INVENTION

The present invention provides for methods of treating cancers with a combination of eribulin, or a pharmaceutical acceptable salt thereof, for example eribulin mesylate, and a selective CDK4/6 inhibitor described herein, wherein the selective CDK4/6 inhibitor is administered prior to administration of eribulin, for example within about 24 hours or less prior to administration of eribulin. It has been discovered that the timely administration of a selective CDK4/6 inhibitor described herein prior to administration of eribulin preserves CDK4/6-replication dependent healthy cells such as hematological cell lines that would otherwise be subject to the myelosuppressive effects of eribulin monotherapy while having no detrimental effect on the antimitotic effects of eribulin against the target cancer cells. Administration of a selective CDK4/6 inhibitor as described herein decreases these effects of eribulin toxicity on CDK 4/6-replication dependent hematological cells, including hematopoietic stem cells and hematopoietic progenitor cells (together referred to as HSPCs) in a subject that is being administered eribulin, providing for a myelopreservation effect. The methods described herein arrest cells in the G1 phase of the cell cycle, making healthy cells resistant to the toxic effects of eribulin as exemplified in FIGS. 2A, 2B and 3, while surprisingly showing no antagonistic effect on the efficacy of eribulin against cancer cells as exemplified in FIGS. 1 and 4, including CDK 4/6-replication dependent cancers.

By incorporating the CDK4/6 inhibitors in a therapeutic regimen described herein, eribulin therapy can be continued for longer periods without requiring dose reduction or cessation of therapy due to toxic side effects. In addition, in certain regimens, administering a combination of eribulin and a CDK4/6 inhibitor described herein provides a surprising synergistic effect as shown in FIG. 4. Furthermore, by incorporating the CDK4/6 inhibitor in a treatment regime described herein that includes eribulin, host immune effector cells may be preserved and immunodepletion reduced, providing for an additional anti-cancer effect through natural lymphocytic attack.

In one aspect, a method is provided for treating cancer in a subject, typically a human, comprising administrating an effective amount of a selective CDK4/6 inhibitor to the subject in combination with administering to the subject eribulin, wherein the CDK4/6 inhibitor is administered to the subject within about 24 hours or less, for example 20, 18, 16, 14, 12, 10, 8, 6, 4, 2, 1, or ¹/₂ hours prior to administration of eribulin. In some embodiments, the CDK4/6 inhibitor is selected from Compound 1-5, palbociclib, abemaciclib, ribociclib, or SHR6390. In some embodiments, the CDK4/6 inhibitor is selected from Compound 1 or Compound 2 or a pharmaceutically acceptable salt, composition, isotopic analog, or prodrug thereof. Compound 1 and Compound 2 have the formulas:

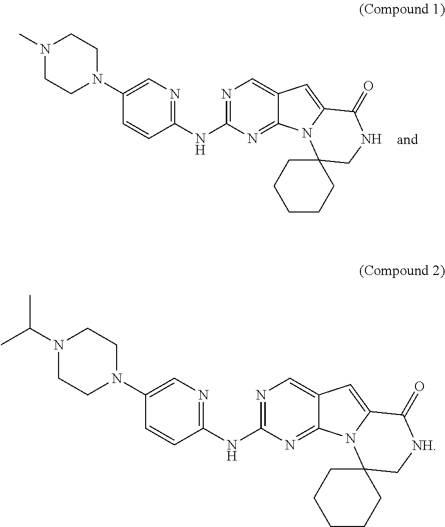 [SMILES: CN1CCN(c2ccc(Nc3ncc4cc5n(c4n3)C3(CCCCC3)CNC5=O)nc2)CC1 ; CC(C)N1CCN(c2ccc(Nc3ncc4cc5n(c4n3)C3(CCCCC3)CNC5=O)nc2)CC1]

In some embodiments, the subject has an Rb-positive cancer or tumor. In some embodiments, the subject has a CDK4/6-replication dependent cancer. In some embodiments, the subject has a CDK4/6-replication independent cancer. In some embodiments, the subject has a cancer selected from metastatic breast cancer, unresectable/metastatic liposarcoma, non-small cell lung cancer, prostate cancer, pancreatic cancer, colorectal cancer, bladder cancer, osteosarcoma, leiomyosarcoma, ovarian cancer, cervical cancer, colon cancer, head and neck cancer, sarcoma, relapsed/refractory rhabdomyosarcoma, non-rhabdomyosarcoma soft tissue sarcoma, Ewing sarcoma, angiosarcoma, epithelioid hemangioendothelioma, and urothelial cell cancer.

In certain embodiments, the cancer is a breast cancer selected from the group consisting of triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor negative breast cancer, ductal carcinoma in situ (OCiS), invasive ductal carcinoma, invasive lobular carcinoma, inflammatory breast cancer, Paget disease of the nipple, phyllodes tumor, and a hormone responsive cancer (e.g., hormone responsive breast cancer).

In some embodiments, an additional agent can be administered in the therapeutic regimen described herein. For example, an anti-hormonal agent can be administered to the subject. Anti-hormonal agents are generally used in hormone receptor positive cancers, including breast, ovarian, cervical, and prostate cancers. Anti-hormonal agents for use in the present invention include, but are not limited to, an estrogen inhibitor including but not limited to a SERM (selective estrogen receptor modulator), a SERD (selective estrogen receptor degrader), a complete estrogen receptor degrader, or another form of partial or complete estrogen antagonist, selective androgen receptor modulator, a selective androgen receptor degrader, a complete androgen receptor degrader, or another form of partial or complete androgen antagonist.

In some embodiments, the subject is administered eribulin or its pharmaceutically acceptable salt on day 1 and day 8 of a 21-day treatment cycle, and a CDK4/6 inhibitor described herein is administered within about 24 hours or less prior to the administration of eribulin, for example, about 24 hours, about 16 hours, about 12 hours, about 8 hours, about 4 hours, about 3 hours, about 2 hours, about 1 hour, or about 30 minutes prior to administration of eribulin. In some embodiments, the subject is administered eribulin or its pharmaceutically acceptable salt on day 1, day 8 and day 15 of a 28-day treatment cycle. In some embodiments, the subject is administered Compound 1 or Compound 2, or a pharmaceutically acceptable salt thereof, less than about 8 hours, less than about 7 hours, less than about 6 hours, less than about 5 hours, less than about 4 hours, less than about 3 hours, less than about 2 hours, less than about 1 hour, or about 30 minutes prior to, administration of eribulin or its pharmaceutically acceptable salt. In some embodiments, the subject is administered a first dose of Compound 1 or Compound 2, or a pharmaceutically acceptable salt thereof, about 24 hours prior to administration of eribulin, and a second dose of Compound 1 or Compound 2 is administered about 4 hours or less prior to administration of eribulin, for example less than about 4 hours, less than about 3 hours, less than about 2 hours, less than about 1 hour, or about 30 minutes prior to, administration of eribulin or its pharmaceutically acceptable salt. In some embodiments, the subject has metastatic breast cancer and has received at least two chemotherapeutic regimens for the treatment of metastatic disease comprising an anthracycline and a taxane drug. In another embodiment, the subject has unresectable or metastatic liposarcoma and has received a prior anthracycline-containing regimen.

In one aspect, a method is provided for the preservation of hematopoietic stem and progenitor cells (HSPCs) in a subject, typically a human, during administration of eribulin, or its pharmaceutically acceptable salt, comprising administrating an effective amount of a selective CDK4/6 to the subject in combination with eribulin or its pharmaceutically acceptable salt, wherein the CDK4/6 inhibitor is administered about 24 hours or less prior to administration of eribulin. In some embodiments, the CDK4/6 inhibitor is selected from Compound 1 and Compound 2 or a pharmaceutically acceptable salt, composition, isotopic analog, or prodrug thereof In some embodiments, the subject has an Rb-positive cancer or tumor. In some embodiments, the subject has a cancer selected from metastatic breast cancer, unresectable/metastatic liposarcoma, non-small cell lung cancer, prostate cancer, pancreatic cancer, colorectal cancer, bladder cancer, osteosarcoma, leiomyosarcoma, ovarian cancer, cervical cancer, colon cancer, head and neck cancer, sarcoma, relapsed/refractory rhabdomyosarcoma, non-rhabdomyosarcoma soft tissue sarcoma, Ewing sarcoma, angiosarcoma, epithelioid hemangioendothelioma, and urothelial cell cancer. In some embodiments, the cancer or tumor is CDK4/6-replication dependent. In some embodiments, the subject is administered eribulin or its pharmaceutically acceptable salt on day 1 and day 8 of a 21-day treatment cycle. In some embodiments, the subject is administered eribulin or its pharmaceutically acceptable salt on day 1, day 8 and day 15 of a 28-day treatment cycle. In some embodiments, the subject is administered Compound 1 or Compound 2, or a pharmaceutically acceptable salt thereof, less than about 8 hours, less than about 7 hours, less than about 6 hours, less than about 5 hours, less than about 4 hours, less than about 3 hours, less than about 2 hours, less than about 1 hour, or about 30 minutes prior to, administration of eribulin or its pharmaceutically acceptable salt. In some embodiments, the subject is administered a first dose of Compound 1 or Compound 2, or a pharmaceutically acceptable salt thereof, about 24 hours prior to administration of eribulin, and a second dose of Compound 1 or Compound 2 is administered about 4 hours or less prior to administration of eribulin, for example less than about 4 hours, less than about 3 hours, less than about 2 hours, less than about 1 hour, or about 30 minutes prior to, administration of eribulin or its pharmaceutically acceptable salt. In some embodiments, the subject has metastatic breast cancer and has received at least two chemotherapeutic regimens for the treatment of metastatic disease comprising an anthracycline and a taxane drug. In another embodiment, the subject has unresectable or metastatic liposarcoma and has received a prior anthracycline-containing regimen.

In one aspect, a method is provided for the myelopreservation of hematologic cells in a subject, typically a human, during administration of eribulin, or its pharmaceutically acceptable salt, comprising administrating an effective amount of a selective CDK4/6 to the subject in combination with eribulin or its pharmaceutically acceptable salt, wherein the CDK4/6 inhibitor is administered about 24 hours or less prior to administration of eribulin. In some embodiments, the CDK4/6 inhibitor is selected from Compound 1 and Compound 2 or a pharmaceutically acceptable salt, composition, isotopic analog, or prodrug thereof. In some embodiments, the subject has an Rb-positive cancer or tumor. In some embodiments, the subject has a cancer selected from metastatic breast cancer, unresectable/metastatic liposarcoma, non-small cell lung cancer, prostate cancer, pancreatic cancer, colorectal cancer, bladder cancer, osteosarcoma, leiomyosarcoma, ovarian cancer, cervical cancer, colon cancer, head and neck cancer, sarcoma, relapsed/refractory rhabdomyosarcoma, non-rhabdomyosarcoma soft tissue sarcoma, Ewing sarcoma, angiosarcoma, epithelioid hemangioendothelioma, and urothelial cell cancer. In some embodiments, the cancer or tumor is CDK4/6-replication dependent. In some embodiments, the subject is administered eribulin or its pharmaceutically acceptable salt on day 1 and day 8 of a 21-day treatment cycle. In some embodiments, the subject is administered eribulin or its pharmaceutically acceptable salt on day 1, day 8 and day 15 of a 28-day treatment cycle. In some embodiments, the subject is administered Compound 1 or Compound 2, or a pharmaceutically acceptable salt thereof, less than about 8 hours, less than about 7 hours, less than about 6 hours, less than about 5 hours, less than about 4 hours, less than about 3 hours, less than about 2 hours, less than about 1 hour, or about 30 minutes prior to, administration of eribulin or its pharmaceutically acceptable salt. In some embodiments, the subject is administered a first dose of Compound 1 or Compound 2, or a pharmaceutically acceptable salt thereof, about 24 hours prior to administration of eribulin, and a second dose of Compound 1 or Compound 2 is administered about 4 hours or less prior to administration of eribulin, for example less than about 4 hours, less than about 3 hours, less than about 2 hours, less than about 1 hour, or about 30 minutes prior to, administration of eribulin or its pharmaceutically acceptable salt. In some embodiments, the subject has metastatic breast cancer and has received at least two chemotherapeutic regimens for the treatment of metastatic disease comprising an anthracycline and a taxane drug. In another embodiment, the subject has unresectable or metastatic liposarcoma and has received a prior anthracycline-containing regimen.

In one aspect, a method is provided for reducing myelosuppression or myeloablation in a subject receiving eribulin, typically a human, comprising administrating an effective amount of a selective CDK4/6 inhibitor described herein to the subject in combination with eribulin or its pharmaceutically acceptable salt, wherein the CDK4/6 inhibitor is administered about 24 hours or less prior to administration of eribulin. In some embodiments, the selective CDK4/6 inhibitor is selected from Compound 1 or Compound 2 or a pharmaceutically acceptable salt, composition, isotopic analog, or prodrug thereof. In some embodiments, the subject has an Rb-positive cancer or tumor. In some embodiments, the subject has a cancer selected from metastatic breast cancer, unresectable/metastatic liposarcoma, non-small cell lung cancer, prostate cancer, pancreatic cancer, colorectal cancer, bladder cancer, osteosarcoma, leiomyosarcoma, ovarian cancer, cervical cancer, colon cancer, head and neck cancer, sarcoma, relapsed/refractory rhabdomyosarcoma, non-rhabdomyosarcoma soft tissue sarcoma, Ewing sarcoma, angiosarcoma, epithelioid hemangioendothelioma, and urothelial cell cancer. In some embodiments, the subject is administered eribulin or its pharmaceutically acceptable salt on day 1 and day 8 of a 21-day treatment cycle. In some embodiments, the subject is administered eribulin or its pharmaceutically acceptable salt on day 1, day 8 and day 15 of a 28-day treatment cycle. In some embodiments, the subject is administered Compound 1 or Compound 2, or a pharmaceutically acceptable salt thereof, less than about 8 hours, less than about 7 hours, less than about 6 hours, less than about 5 hours, less than about 4 hours, less than about 3 hours, less than about 2 hours, less than about 1 hour, or about 30 minutes prior to, administration of eribulin or its pharmaceutically acceptable salt. In some embodiments, the subject is administered a first dose of Compound 1 or Compound 2, or a pharmaceutically acceptable salt thereof, about 24 hours prior to administration of eribulin, and a second dose of Compound 1 or Compound 2 is administered about 4 hours or less prior to administration of eribulin, for example less than about 4 hours, less than about 3 hours, less than about 2 hours, less than about 1 hour, or about 30 minutes prior to, administration of eribulin or its pharmaceutically acceptable salt. In some embodiments, the subject has metastatic breast cancer and has received at least two chemotherapeutic regimens for the treatment of metastatic disease comprising an anthracycline and a taxane drug. In another embodiment, the subject has unresectable or metastatic liposarcoma and has received a prior anthracycline-containing regimen.

In another aspect, a method is provided for the treatment of an Rb-positive cancer or tumor in a subject, typically a human, comprising administering a selective CDK4/6 inhibitor described herein in combination with eribulin or its pharmaceutically acceptable salt, wherein the CDK4/6 inhibitor is administered about 24 hours or less prior to administration of eribulin. In some embodiments, the selective CDK4/6 inhibitor is Compound 1, Compound 2, or a pharmaceutically acceptable salt, composition, isotopic analog, or prodrug thereof. In some embodiments, the subject has an Rb-positive cancer or tumor, for example a cancer selected from metastatic breast cancer, unresectable/metastatic liposarcoma, non-small cell lung cancer, prostate cancer, pancreatic cancer, colorectal cancer, bladder cancer, osteosarcoma, leiomyosarcoma, ovarian cancer, cervical cancer, colon cancer, head and neck cancer, sarcoma, relapsed/refractory rhabdomyosarcoma, non-rhabdomyosarcoma soft tissue sarcoma, Ewing sarcoma, angiosarcoma, epithelioid hemangioendothelioma, and urothelial cell cancer. In some embodiments, the subject is administered eribulin or its pharmaceutically acceptable salt on day 1 and day 8 of a 21-day treatment cycle. In some embodiments, the subject is administered eribulin or its pharmaceutically acceptable salt on day 1, day 8 and day 15 of a 28-day treatment cycle. In some embodiments, the subject is administered Compound 1 or Compound 2, or a pharmaceutically acceptable salt thereof, concomitantly or prior to, for example less than about 8 hours, less than about 7 hours, less than about 6 hours, less than about 5 hours, less than about 4 hours, less than about 3 hours, less than about 2 hours, less than about 1 hour, or about 30 minutes prior to, administration of eribulin or its pharmaceutically acceptable salt. In some embodiments, the subject is administered a first dose of Compound 1 or Compound 2, or a pharmaceutically acceptable salt thereof, about 24 hours prior to administration of eribulin, and a second dose of Compound 1 or Compound 2 is administered about 4 hours or less prior to administration of eribulin. In some embodiments, the subject has metastatic breast cancer and has received at least two chemotherapeutic regimens for the treatment of metastatic disease comprising an anthracycline and a taxane drug. In another embodiment, the subject has unresectable or metastatic liposarcoma and has received a prior anthracycline-containing regimen.

In an alternative embodiment, a method is provided for the treatment of a cancer or tumor in a subject, typically a human, comprising administering a selective CDK4/6 inhibitor described herein in combination with eribulin or its pharmaceutically acceptable salt, wherein the CDK4/6 inhibitor is administered at least once a day on days 1-21 of a 21-day treatment cycle, and wherein the subject is administered eribulin or its pharmaceutically acceptable salt, e.g., eribulin mesylate, on day 1 and day 8 of a 21-day treatment cycle. In some embodiments, the CDK4/6 inhibitor is Compound 1, or a pharmaceutically acceptable salt composition, isotopic analog, or prodrug thereof. In some embodiments, the selective CDK4/6 inhibitor is Compound 2, or a pharmaceutically acceptable salt, e.g., dihydrochloride salt, composition, isotopic analog, or prodrug thereof. In some embodiments, the cancer or tumor is a CDK4/6-replication dependent cancer or tumor. In some embodiments, the cancer or tumor is a CDK4/6-replication independent cancer or tumor. In some embodiments, the subject has an Rb-positive cancer or tumor. In some embodiments, the subject has a cancer selected from metastatic breast cancer, unresectable/metastatic liposarcoma, non-small cell lung cancer, prostate cancer, pancreatic cancer, colorectal cancer, bladder cancer, osteosarcoma, leiomyosarcoma, ovarian cancer, cervical cancer, colon cancer, head and neck cancer, sarcoma, relapsed/refractory rhabdomyosarcoma, non-rhabdomyosarcoma soft tissue sarcoma, Ewing sarcoma, angiosarcoma, epithelioid hemangioendothelioma, and urothelial cell cancer. In some embodiments, the subject is administered Compound 2, or a pharmaceutically acceptable salt thereof, e.g., dihydrochloride salt, concomitantly or prior to, for example less than about 8 hours, less than about 7 hours, less than about 6 hours, less than about 5 hours, less than about 4 hours, less than about 3 hours, less than about 2 hours, less than about 1 hour, or about 30 minutes prior to, administration of eribulin or its pharmaceutically acceptable salt, e.g., eribulin mesylate. In some embodiments, the subject has metastatic breast cancer and has received at least two chemotherapeutic regimens for the treatment of metastatic disease comprising an anthracycline and a taxane drug. In another embodiment, the subject has unresectable or metastatic liposarcoma and has received a prior anthracycline-containing regimen. In certain embodiments, methods of the present invention can also include the administration of one or more additional therapeutic agents. In some embodiments, the additional therapeutic agent is an anti-hormonal agent, for example a SERM (selective estrogen receptor modulator), a SERD (selective estrogen receptor degrader), a complete estrogen receptor degrader, or another form of partial or complete estrogen antagonist, selective androgen receptor modulator, a selective androgen receptor degrader, a complete androgen receptor degrader, or another form of partial or complete androgen antagonist. In some embodiments, the additional anti-hormonal agent is selected from fulvestrant, tamoxifen, anastrozole, letrozole, exemestane, goserelin, leuprolide, megestrol acetate and toremifene.

In an alternative embodiment, a method is provided for the treatment of a cancer or tumor in a subject, typically a human, comprising administering a selective CDK4/6 inhibitor described herein in combination or alternation with eribulin or its pharmaceutically acceptable salt, e.g., eribulin mesylate, wherein the CDK4/6 inhibitor is administered at least once daily on days 1-28 of a 28-day treatment cycle whereas the subject is administered eribulin or its pharmaceutically acceptable salt on day 1, day 8 and day 15 of a 28-day treatment cycle. In some embodiments, the selective CDK4/6 inhibitor is Compound 1. In some embodiments, the selective CDK4/6 inhibitor is Compound 2, or a pharmaceutically acceptable salt, e.g., dihydrochloride salt, composition, isotopic analog, or prodrug thereof. In some embodiments, the cancer or tumor is a CDK4/6-replication dependent cancer or tumor. In some embodiments, the cancer or tumor is a CDK4/6-replication independent cancer or tumor. In some embodiments, the subject has an Rb-positive cancer or tumor. In some embodiments, the subject has a cancer selected from metastatic breast cancer, unresectable/metastatic liposarcoma, non-small cell lung cancer, prostate cancer, pancreatic cancer, colorectal cancer, bladder cancer, osteosarcoma, leiomyosarcoma, ovarian cancer, cervical cancer, colon cancer, head and neck cancer, sarcoma, relapsed/refractory rhabdomyosarcoma, non-rhabdomyosarcoma soft tissue sarcoma, Ewing sarcoma, angiosarcoma, epithelioid hemangioendothelioma, and urothelial cell cancer. In some embodiments, the subject is administered Compound 2, or a pharmaceutically acceptable salt thereof, e.g., dihydrochloride salt, concomitantly or prior to, for example less than about 8 hours, less than about 7 hours, less than about 6 hours, less than about 5 hours, less than about 4 hours, less than about 3 hours, less than about 2 hours, less than about 1 hour, or about 30 minutes prior to, administration of eribulin or its pharmaceutically acceptable salt e.g., eribulin mesylate. In some embodiments, the subject has metastatic breast cancer and has received at least two chemotherapeutic regimens for the treatment of metastatic disease comprising an anthracycline and a taxane drug. In another embodiment, the subject has unresectable or metastatic liposarcoma and has received a prior anthracycline-containing regimen. In some embodiments, the additional therapeutic agent is an anti-hormonal agent, for example a SERM (selective estrogen receptor modulator), a SERD (selective estrogen receptor degrader), a complete estrogen receptor degrader, or another form of partial or complete estrogen antagonist, selective androgen receptor modulator, a selective androgen receptor degrader, a complete androgen receptor degrader, or another form of partial or complete androgen antagonist. In some embodiments, the additional anti-hormonal agent is selected from fulvestrant, tamoxifen, anastrozole, letrozole, exemestane, goserelin, leuprolide, megestrol acetate and toremifene.

### BRIEF DESCRIPTION OF THE FIGURES

FIG. 1 is a line graph that shows the effect on tumor growth of treatment of mice bearing the MD-MB-231 xenograft model with Compound 1 (100 mg/kg, IP), eribulin (IV, 0.5 mg/kg), Compound 1 (100 mg/kg, IP) and eribulin (100 mg/kg, IV), or vehicle for three weeks, where Compound 1 was administered 30 minutes prior to eribulin. The graphs represent mean tumor volume over time. The y-axis is tumor volume measured in cubic millimeters. The x-axis is time measured in days. The dotted line represents dates of weekly treatment. The error bars represent standard error of the mean.

FIG. 2A is a graph depicting the percentage of EdU positive MCF7 tumor cells vs. Edu positive Lin⁻ bone marrow cells from MCF7 tumor bearing mice treated with Compound 1 at 4, 12, 24, and 48 hours post treatment. MCF7 tumor cells are on the left at each time point while Lin- bone marrow cells are on the right at each time point. FIG. 2B is a graph depicting the percentage of EdU positive MCF7 tumor cells vs. Edu positive Lin⁻ bone marrow cells from MCF7 tumor bearing mice treated with Compound 1 at 4, 12, 24, and 48 hours post treatment normalized to baseline. MCF7 tumor cells are on the left at each time point while Lin- bone marrow cells are on the right at each time point.

FIG. 3 is a graph depicting the differences in baseline proliferation rates of hematopoietic stem and progenitor (HSPCs), total bone marrow, and PDX tumors cells examined using flow cytometric analysis of the cell cycle. The bar graph depicts mean percentage of cells in S/G2/M phase of the cell cycle.

FIG. 4 is a line graph that shows the effect on tumor growth of treatment of mice bearing the MDA-MB-231 xenograft model with Compound 1 (IP, 100 mg/kg, n=10, dosed daily for 28 days); eribulin (IV, 0.5 mg/kg, n=10, dosed every 3 weeks); Compound 1 (IP, 100 mg/kg, n=10, dosed daily for 28 days) and eribulin (IV, 0.5 mg/kg, n=10, dosed every 3 weeks); and vehicle (IP, n=10, dosed every 3 weeks).The graphs represent mean tumor volume over time. The y-axis is tumor volume measured in cubic millimeters. The x-axis is time measured in days. The dotted line represents dates of weekly treatment. The error bars represent standard error of the mean.

### DETAILED DESCRIPTION OF THE INVENTION

### Definitions

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. Recitation of ranges of values are merely intended to serve as a shorthand method of referring individually to each separate value falling within the range, unless otherwise indicated herein, and each separate value is incorporated into the specification as if it were individual recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and do not pose a limitation on the scope of the invention unless otherwise claimed. Unless defined otherwise, technical and scientific terms used herein have the same meaning as is commonly understood by one of skill in the art to which this invention belongs.

In non-limiting embodiments, Compound 1, Compound 2, or eribulin can be used in a form that has at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into Compound 1, Compound 2, or eribulin for use in the invention include isotopes of hydrogen, carbon, nitrogen, oxygen, and sulfur such as ²H, ³H, ¹¹C, ¹³C, ¹⁴C, ¹⁵N, and ³⁵S. In one non-limiting embodiment, isotopically labelled compounds can be used in metabolic studies (with ¹⁴C), reaction kinetic studies (with, for example ²H and ³H), detection or imagine techniques, such as positron emission tomography (PET) or single-photon emission computed tomography (SPECT) including drug or substrate tissue distribution assays, or in radioactive treatment of patients. Isotopically labeled compounds of this invention and prodrugs thereof can generally be prepared by carrying out the procedures disclosed in the schemes or in the examples and preparations described below by substituting a readily available isotopically labeled reagent for non-isotopically labeled reagent.

By way of general example and without limitation, isotopes of hydrogen, for example, deuterium (²H) and tritium (³H) may be used anywhere in described structures that achieves the desired result. Alternatively, or in addition, isotopes of carbon, e.g., ¹³C and ¹⁴C, may be used. Isotopic substitutions, for example deuterium substitutions, can be partial or complete.

Partial deuterium substitution means that at least one hydrogen is substituted with deuterium. In certain embodiments, the molecule is 90, 95, or 99% or more enriched in an isotope at any location of interest. In one non-limiting embodiment, deuterium is 90, 95, or 99% enriched at a desired location. Compound 1, Compound 2, or eribulin for use in the present invention may form a solvate with solvents (including water). Therefore, in one non-limiting embodiment, the invention includes a solvated form of the compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a compound of the invention and water. Pharmaceutically acceptable solvates in accordance with the invention include those wherein the solvent may be isotopically substituted, e.g. D₂O, d₆-acetone, d₆-DMSO. A solvate can be in a liquid or solid form.

As generally contemplated herein, the term hematopoietic stem and progenitor cell (HSPC) includes, but are not limited to, long term hematopoietic stem cells (LT-HSCs), short term hematopoietic stem cells (ST-HSCs), hematopoietic progenitor cells (HPCs), multipotent progenitors (MPPs), oligodendrocyte pre-progenitors (OPPs), monocyte progenitors, granulocyte progenitors, common myeloid progenitors (CMPs), common lymphoid progenitors (CLPs), granulocyte-monocyte progenitors (GMPs), granulocyte progenitors, monocyte progenitors, and megakaryocyte-erythroid progenitors (MEPs), megakaryocyte progenitors, erythroid progenitors, HSC/MPPs (CD45dim/CD34+/CD38−), OPPs (CD45dim/CD34+/CD38+), monocyte progenitors (CD45+/CD14+/CD11b+), granulocyte progenitors (CD45+/CD14−/CD11b+), erythroid progenitors (CD45−/CD71+), and megakaryocyte progenitors (CD45+/CD61+).

The subject treated is typically a human subject, although it is to be understood the methods described herein are effective with respect to other animals, such as mammals and vertebrate species. More particularly, the term subject can include animals used in assays such as those used in preclinical testing including but not limited to mice, rats, monkeys, dogs, pigs and rabbits; as well as domesticated swine (pigs and hogs), ruminants, equine, poultry, felines, bovines, murines, canines, and the like.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

In some embodiments, the term “CDK4/6-replication dependent cancer” refers to a cancer or cellular proliferation disorder that requires the activity of CDK4/6 for replication or proliferation, or which may be growth inhibited through the activity of a selective CDK4/6 inhibitor. Cancers and disorders of such type can be characterized by (e.g., that has cells that exhibit) the presence of a functional Retinoblastoma protein. Such cancers and disorders are classified as being Rb-positive.

The term “selective CDK4/6 inhibitor” used in the context of the compounds described herein includes compounds that inhibit CDK4 activity, CDK6 activity, or both CDK4 and CDK6 activity at an IC50 molar concentration at least about 100, 200, 300, 400, or 500 times less (or in alternative embodiments, at least 750, 1000, 1500, or 2000 times less) than the IC50 molar concentration necessary to inhibit to the same degree of CDK2 activity in a standard phosphorylation assay.

In some embodiments, the term “CDK4/6-replication independent cancer” refers to a cancer that does not significantly require the activity of CDK4/6 for replication. Cancers of such type are often, but not always, characterized by (e.g., that has cells that exhibit) an increased level of CDK2 activity or by reduced expression of retinoblastoma tumor suppressor protein or retinoblastoma family member protein(s), such as, but not limited to p107 and p130. The increased level of CDK2 activity or reduced or deficient expression of retinoblastoma tumor suppressor protein or retinoblastoma family member protein(s) can be increased or reduced, for example, compared to normal cells. In some embodiments, the increased level of CDK2 activity can be associated with (e.g., can result from or be observed along with) MYC protooncogene amplification or overexpression. In some embodiments, the increased level of CDK2 activity can be associated with overexpression of Cyclin E1, Cyclin E2, or Cyclin A.

An “effective amount” as used herein, means an amount which provides a therapeutic or prophylactic benefit.

To “treat” a disease as the term is used herein, means to reduce the frequency or severity of at least one sign or symptom of a disease or disorder experienced by a subject (i.e. palliative treatment) or to decrease a cause or effect of the disease or disorder (i.e. disease-modifying treatment).

Throughout this disclosure, various aspects of the invention can be presented in a range format. It should be understood that the description in range format is merely for convenience and should not be construed as a limitation on the scope of the invention. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, “pharmaceutical compositions” are compositions comprising at least one active agent, and at least one other substance, such as a carrier. “Pharmaceutical combinations” are combinations of at least two active agents which may be combined in a single dosage form or provided together in separate dosage forms with instructions that the active agents are to be used together to treat any disorder described herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions/combinations of the invention refers to a diluent, excipient, or vehicle with which an active compound is provided.

### Selective CDK4/6 Inhibitors

Selective CDK4/6 inhibitors for use in the present invention include Compound 1 or Compound 2, or pharmaceutically acceptable salts thereof.

Compound 1, also known as trilaciclib (2′-((5-(4-methylpiperazin-1-yl)pyridin-2-yl)amino)-7′,8′-dihydro-6′H-spiro(cyclohexane-1,9′-pyrazino(1′,2′: 1,5)pyrrolo(2,3 -d)pyrimidin)-6′-one) is a highly selective CDK4/6 inhibitor having the structure:

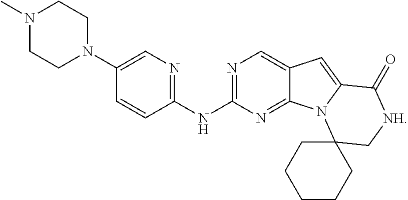 [SMILES: CN1CCN(c2ccc(Nc3ncc4cc5n(c4n3)C3(CCCCC3)CNC5=O)nc2)CC1]

As provided herein, Compound 1 or its pharmaceutically acceptable salt, composition, isotopic analog, or prodrug thereof is administered in a suitable carrier in a therapeutic regime that includes eribulin or its pharmaceutically acceptable salt thereof. Compound 1 is described in U.S. Pat. No. 8,598,186, incorporated herein by reference in its entirety. Compound 1 can be synthesized as described in WO 2019/0135820, incorporated herein by reference in its entirety.

Compound 1 can be intravenously administered to a patient prior to administration of eribulin or its pharmaceutically acceptable salt thereof. In some embodiments, Compound 1 is administered about 4 hours or less, for example about 30-60 minutes or less, prior to administration of eribulin or its pharmaceutically acceptable salt. In some embodiments, Compound 1 is administered once approximately around 24 hours, for example about 22 to 26 hours, before administration of eribulin or its pharmaceutically acceptable salt thereof, and again about 4 hours or less, for example about 30-60 minutes or less, prior to administration of eribulin or its pharmaceutically acceptable salt thereof. In some embodiments, the dose of Compound 1 administered is between about 180 and about 280 mg/m2. For example, the dose is about 180, 185, 190, 195, 200, 205, 210, 215, 220, 225, 230, 235, 240, 245, 250, 255, 260, 265, 270, 275, or 280 mg/m² or any dose in between these numbers as determined desirable by the healthcare practitioner. In a particular embodiment, the dose is about 240 mg/m².

In an alternative embodiment, Compound 2, known as lerociclib, or its pharmaceutically acceptable salt, is administered. Compound 2 (2′-((5-(4-isopropylpiperazin-1-yl) pyridin-2-yl)amino)-7′,8′-dihydro-6′H-spiro[cyclohexane-1,9′-pyrazino[1′,2′:1,5]pyrrolo[2,3-d]pyrimidin]-6′-one) has the chemical structure:

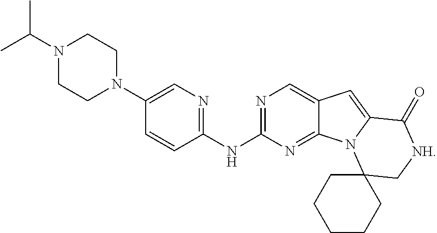 [SMILES: CC(C)N1CCN(c2ccc(Nc3ncc4cc5n(c4n3)C3(CCCCC3)CNC5=O)nc2)CC1]

Compound 2 can be administered orally or intravenously. Compound 2 can be prepared as previously described in WO 2014/144325, incorporated herein by reference. In one embodiment, Compound 2 is administered as the dihydrochloride salt. In one embodiment, the Form B morphic form of the dihydrochloride salt as described in WO 2019/006393, incorporated herein by reference in its entirety.

Another selective CDK4/6 inhibitor for use in the present invention includes the CDK4/6 inhibitor having the structure:

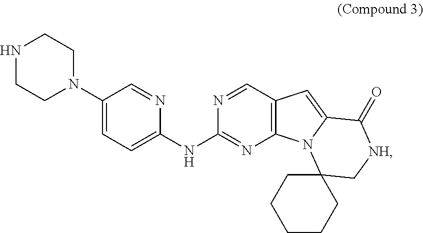 [SMILES: O=C1NCC2(CCCCC2)n2c1cc1cnc(Nc3ccc(N4CCNCC4)cn3)nc12]

or its pharmaceutically acceptable salt. Compound 3 can be administered orally or intravenously. Compound 3 can be prepared as previously described in WO 2014/144325, incorporated herein by reference.

Another selective CDK4/6 inhibitor for use in the present invention includes the CDK4/6 inhibitor having the structure:

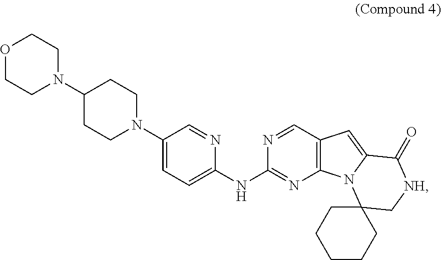 [SMILES: O=C1NCC2(CCCCC2)n2c1cc1cnc(Nc3ccc(N4CCC(N5CCOCC5)CC4)cn3)nc12]

or its pharmaceutically acceptable salt. Compound 4 can be administered orally or intravenously. Compound 4 can be prepared as previously described in WO 2014/144325, incorporated herein by reference.

Another selective CDK4/6 inhibitor for use in the present invention includes the CDK4/6 inhibitor having the structure:

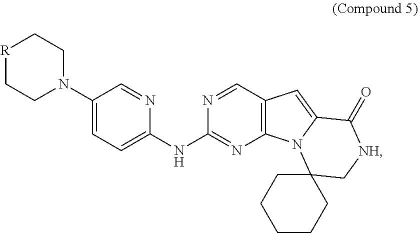

wherein R is C(H)X, NX, C(H)Y, or C(X)₂,

where X is straight, branched or cyclic C₁ to C₅ alkyl group, including methyl, ethyl, propyl, cyclopropyl, isopropyl, butyl, sec-butyl, tert-butyl, isobutyl, cyclobutyl, pentyl, isopentyl, neopentyl, tert-pentyl, sec-pentyl, and cyclopentyl; and

Y is NR₁R₂ wherein R₁ and R₂ are independently X, or wherein R₁ and R₂ are alkyl groups that together form a bridge that includes one or two heteroatoms (N, O, or S);

and wherein two X groups can together form an alkyl bridge or a bridge that includes one or two heteroatoms (N, S, or O) to form a spiro compound, or its pharmaceutically acceptable salt. Compound 5 can be administered orally or intravenously. Compound 5 can be prepared as previously described in WO 2014/144325, incorporated herein by reference.

In yet another alternative embodiment, the CDK4/6 inhibitor is selected from palbociclib, abemaciclib, ribociclib, or SHR6390.

### Eribulin

Eribulin is a synthetic analogue of halichondrin B, a product isolated from the marine sponge Halichondria okadai. Eribulin has the structure:

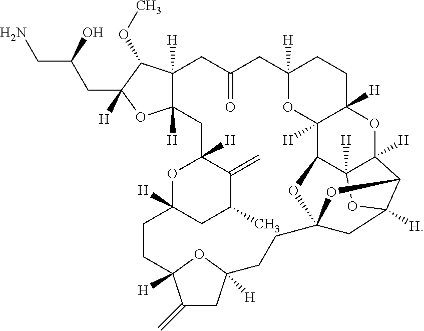

Eribulin mesylate has the following structural formula:

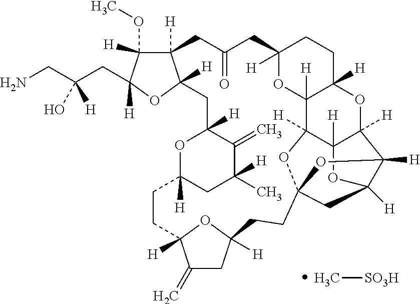

The chemical name for eribulin mesylate is 11,15:18,21:24,28Triepoxy-7,9-ethano-12,15-methano-9H,15H-furo[3,2-i]furo[2′,3′:5,6]pyrano[4,3-b][1,4]dioxacyclopentacosin-5(4H)-one, 2-[(2S)-3amino-2-hydroxypropyl]hexacosahydro-3-methoxy-26-methyl-20,27-bis(methylene)-, (2R,3R,3aS,7R,8aS,9S,10aR,11S,12R,13aR,13bS,15S,18S,21S,24S,26R,28R,29aS)-, methanesulfonate (salt). It has a molecular weight of 826.0 (729.9 for free base). The empirical formula is C₄₀H₅₉NO₁₁.CH₄O₃S.

Methods for synthesis of eribulin are described, for example, in U.S. Pat. No. 6,214,865; U.S. Pat. No. 7,982,060; U.S. Pat. No. 8,350,067; and U.S. Pat. No. 8,093,410, each of which is incorporated herein by reference. Eribulin mesylate is available commercially and is marketed as Halaven™.

Halaven™ is indicated for the treatment of patients with metastatic breast cancer who have previously received at least two chemotherapeutic regimens for the treatment of metastatic disease. Prior therapy should have included an anthracycline and a taxane in either the adjuvant or metastatic setting. Halaven™ is also indicated for the treatment of patients with unresectable or metastatic liposarcoma who have received a prior anthracycline-containing regimen.

### Anti-Hormonal Agents

In some embodiments, an additional agent can be administered in the eribulin therapeutic regimen described herein. For example, an anti-hormonal agent can be administered to the subject. Anti-hormonal agents are generally used in hormone receptor positive cancers, including breast, ovarian, cervical, and prostate cancers.

Anti-hormonal agents for use in the present invention include, but are not limited to, an estrogen inhibitor including but not limited to a SERM (selective estrogen receptor modulator), a SERD (selective estrogen receptor degrader), a complete estrogen receptor degrader, or another form of partial or complete estrogen antagonist, selective androgen receptor modulator, a selective androgen receptor degrader, a complete androgen receptor degrader, or another form of partial or complete androgen antagonist.

Partial anti-estrogens like raloxifene and tamoxifen retain some estrogen-like effects, including an estrogen-like stimulation of uterine growth, and also, in some cases, an estrogen-like action during breast cancer progression which actually stimulates tumor growth. In contrast, fulvestrant, a complete anti-estrogen, is free of estrogen-like action on the uterus and is effective in tamoxifen-resistant tumors. Non-limiting examples of anti-estrogen compounds are provided in WO2014/19176 assigned to Astra Zeneca, WO2013/090921, WO2014/203129, WO 2014/203132, and US2013/0178445 assigned to Olema Pharmaceuticals, and U.S. Pat. Nos. 9,078,871, 8,853,423, and 8,703,810, as well as US 2015/0005286, WO 2014/205136, and WO 2014/205138.

Additional non-limiting examples of anti-estrogen compounds include: SERMS such as anordrin, bazedoxifene, broparestriol, chlorotrianisene, clomiphene citrate, cyclofenil, lasofoxifene, ormeloxifene, raloxifene, tamoxifen, toremifene, and fulvestrant; aromatase inhibitors such as aminoglutethimide, testolactone, anastrozole, exemestane, fadrozole, formestane, and letrozole; and antigonadotropins such as leuprorelin, cetrorelix, allylestrenol, chloromadinone acetate, cyproterone acetate, delmadinone acetate, dydrogesterone, medroxyprogesterone acetate, megestrol acetate, nomegestrol acetate, norethisterone acetate, progesterone, and spironolactone. Other estrogenic ligands that can be used according to the present invention are described in U.S. Pat. Nos. 4,418,068; 5,478,847; 5,393,763; and 5,457,117, WO2011/156518, U.S. Pat. Nos. 8,455,534 and 8,299,112, U.S. Pat. Nos. 9,078,871; 8,853,423; 8,703,810; US 2015/0005286; and WO 2014/205138, US2016/0175289, US2015/0258080, WO 2014/191726, WO 2012/084711; WO 2002/013802; WO 2002/004418; WO 2002/003992; WO 2002/003991; WO 2002/003990; WO 2002/003989; WO 2002/003988; WO 2002/003986; WO 2002/003977; WO 2002/003976; WO 2002/003975; WO 2006/078834; US 6821989; US 2002/0128276; U.S. Pat. No. 6,777,424; US 2002/0016340; U.S. Pat. No. 6,326,392; U.S. Pat. No. 6,756,401; US 2002/0013327; U.S. Pat. No. 6,512,002; U.S. Pat. No. 6,632,834; US 2001/0056099; U.S. Pat. No. 6,583,170; U.S. Pat. No. 6,479,535; WO 1999/024027; U.S. Pat. No. 6,005,102; EP 0802184; U.S. Pat. No. 5,998,402; U.S. Pat. No. 5,780,497, U.S. Pat. No. 5,880,137, WO 2012/048058 and WO 2007/087684. Additional SERDs for use in the present invention include the SERD described in WO 2017/100712, WO 2017/100715, US 2017/0166550, or US 2017/0166551.

Nonlimiting examples of anti-androgen compounds are provided in WO 2011/156518 and U.S. Pat. Nos. 8,455,534 and 8,299,112. Additional non-limiting examples of anti-androgen compounds include: chlormadinone acetate, spironolactone, canrenone, drospirenone, ketoconazole, topilutamide, abiraterone acetate, and cimetidine.

### Pharmaceutical Compositions and Dosage Forms

The active compounds described herein for use in the methods described herein, or its salt, isotopic analog, or prodrug can be administered in an effective amount to a subject using any suitable approach which achieves the desired therapeutic result. The amount and timing of the active compounds administered will, of course, be dependent on the subject being treated, the instructions of the supervising medical specialist, on the time course of the exposure, on the manner of administration, on the pharmacokinetic properties of the particular active compound, and on the judgment of the prescribing physician. Thus, because of host to host variability, the dosages given below are a guideline and the physician can titrate doses of the active compounds to achieve the treatment that the physician considers appropriate for the host. In considering the degree of treatment desired, the physician can balance a variety of factors such as age and weight of the host, presence of preexisting disease, as well as presence of other diseases. General administration dosages for selective CDK4/6 inhibitors such as Compound 1 have been previously described in WO 2016/126889, incorporated herein by its entirety.

The pharmaceutical composition may be formulated as any pharmaceutically useful form, e.g., a pill, an injection or infusion solution, a capsule, a tablet, a syrup, a dry powder, an inhalation formulation, suppository, buccal, or sublingual formulation, or parenteral formulation. Some dosage forms, such as tablets and capsules, are subdivided into suitably sized unit doses containing appropriate quantities of the active components, e.g., an effective amount to achieve the desired purpose.

The therapeutically effective dosage of any active compound described herein will be determined by the health care practitioner depending on the condition, size and age of the patient as well as the route of delivery. In one non-limited embodiment, a dosage from about 0.1 to about 200 mg/kg has therapeutic efficacy, with all weights being calculated based upon the weight of the active compound, including the cases where a salt is employed. In some embodiments, the dosage may be the amount of compound needed to provide a serum concentration of the active compound of up to about 10 nM, 50 nM, 100 nM, 200 nM, 300 nM, 400 nM, 500 nM, 600 nM, 700 nM, 800 nM, 900 nM, 1 μM, 5 μM, 10 μM, 20 μM, 30 μM, or 40 μM.

In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of the active compound and optionally from about 0.1 mg to about 2000 mg, from about 10 mg to about 1000 mg, from about 100 mg to about 800 mg, or from about 200 mg to about 600 mg of an additional active agent in a unit dosage form. Examples of dosage forms with at least 5, 10, 15, 20, 25, 50, 100, 200, 250, 300, 400, 500, 600, 700, or 750 mg of active compound, or its salt. The pharmaceutical composition may also include a molar ratio of the active compound and an additional active agent, in a ratio that achieves the desired results.

In some embodiments, the selective CDK4/6 inhibitor administered is Compound 1, or its pharmaceutically acceptable salt, which is administered at a dosage of about 180 mg/m² to about 280 mg/m². In some embodiments, Compound 1 is administered at about 180, 185, 190, 195, 200, 205, 210, 215, 220, 225, 230, 235, 240, 245, 250, 255, 260, 265, 270, 275, or about 280 mg/m². In some embodiments, Compound 1 is administered at a dose of about 200 mg/m². In some embodiments, Compound 1 is administered at a dose of about 240 mg/m².

In some embodiments, the selective CDK4/6 inhibitor administered is Compound 2, or its pharmaceutically acceptable salt, which is administered at a dosage of between about 100 mg to about 250 mg. In some embodiments, Compound 2 is administered at about 100, 125, 150, 180, 185, 190, 195, 200, 205, 210, 215, 220, 225, 230, 235, 240, 245, 250, 255, 260, 265, 270, 275, or about 280 mg. In some embodiments, Compound 2 is administered at a dose of about 150 mg. In some embodiments, Compound 2 is administered at a dose of about 200 mg. In some embodiments, Compound 2 is administered at a dose of about 250 mg.

In some embodiments, eribulin, or its pharmaceutically acceptable salt, e.g., eribulin mesylate, is administered at a dosage of about 0.5 to about 1.5 mg/m² over 2 to 5 minutes. In some embodiments, eribulin or its pharmaceutically acceptable salt, e.g., eribulin mesylate, is administered at about 0.5, 0.7, 1.0, 1.1, 1.4, or about 1.5 mg/m². In some embodiments, eribulin or its pharmaceutically acceptable salt, e.g., eribulin mesylate, is administered at a dose of about 0.7 mg/m². In some embodiments, eribulin or its pharmaceutically acceptable salt, e.g., eribulin mesylate, is administered at a dose of about 1.1 mg/m². In some embodiments, eribulin or its pharmaceutically acceptable salt, e.g., eribulin mesylate, is administered at a dose of about 1.4 mg/m².

Compounds disclosed herein or used as described herein may be administered orally, topically, parenterally, by inhalation or spray, sublingually, via implant, transdermally, via buccal administration, rectally, intravenous, intramuscular, inhalation, intra-aortal, intracranial, subdermal, intraperitoneal, subcutaneous, transnasal, sublingual, or rectal or by other means, in dosage unit formulations containing conventional pharmaceutically acceptable carriers.

The pharmaceutical formulations can comprise an active compound described herein or a pharmaceutically acceptable salt thereof, in any pharmaceutically acceptable carrier. If a solution is desired, water may sometimes be the carrier of choice for water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and for illustration by filtration through a 0.22-micron filter. Subsequent to sterilization, the solution can be dispensed into appropriate receptacles, such as depyrogenated glass vials. The dispensing is optionally done by an aseptic method. Sterilized closures can then be placed on the vials and, if desired, the vial contents can be lyophilized.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

### Cancer or Tumor Types

As contemplated herein, the use of a selective CDK4/6 inhibitor or its pharmaceutically acceptable salt in combination with eribulin or its pharmaceutically acceptable salt can be used in the treatment of a subject having a cancer or tumor. In some embodiments, the cancer or tumor is a CDK4/6-replication dependent cancer or tumor. In some embodiments, the cancer or tumor is a CDK4/6-replication independent cancer or tumor.

In particular embodiments, the methods described herein can be used to treat a subject with a Rb-positive cancer. In some embodiments, the cancer is a CDK4/6-replication dependent cancer, which refers to a cancer that requires the activity of CDK4/6 for replication or proliferation, or which may be growth inhibited through the activity of a selective CDK4/6 inhibitor. Cancers and disorders of such type can be characterized by (e.g., that has cells that exhibit) the presence of a functional Retinoblastoma protein. Such cancers and disorders are classified as being Rb-positive.

Targeted cancers suitable for administration of a the combination of compounds described herein may include Rb-positive: estrogen-receptor positive cancer (ER+), HER2-negative advanced breast cancer, late-line metastatic breast cancer, liposarcoma, non-small cell lung cancer, liver cancer, ovarian cancer, glioblastoma, refractory solid tumors, retinoblastoma positive breast cancer as well as retinoblastoma positive endometrial, vaginal and ovarian cancers and lung and bronchial cancers, adenocarcinoma of the colon, adenocarcinoma of the rectum, central nervous system germ cell tumors, teratomas, estrogen receptor-negative breast cancer, estrogen receptor-positive breast cancer, familial testicular germ cell tumors, HER2-negative breast cancer, HER2-positive breast cancer, male breast cancer, ovarian immature teratomas, ovarian mature teratoma, ovarian monodermal and highly specialized teratomas, progesterone receptor-negative breast cancer, progesterone receptor-positive breast cancer, recurrent breast cancer, recurrent colon cancer, recurrent extragonadal germ cell tumors, recurrent extragonadal non-seminomatous germ cell tumor, recurrent extragonadal seminomas, recurrent malignant testicular germ cell tumors, recurrent melanomas, recurrent ovarian germ cell tumors, recurrent rectal cancer, stage III extragonadal non-seminomatous germ cell tumors, stage III extragonadal seminomas, stage III malignant testicular germ cell tumors, stage III ovarian germ cell tumors, stage IV breast cancers, stage IV colon cancers, stage IV extragonadal non-seminomatous germ cell tumors, stage IV extragonadal seminoma, stage IV melanomas, stage IV ovarian germ cell tumors, stage IV rectal cancers, testicular immature teratomas, testicular mature teratomas. In particular embodiments, the targeted cancers included estrogen-receptor positive, HER2-negative advanced breast cancer, late-line metastatic breast cancer, liposarcoma, non-small cell lung cancer, liver cancer, ovarian cancer, glioblastoma, refractory solid tumors, retinoblastoma positive breast cancer as well as retinoblastoma positive endometrial, vaginal and ovarian cancers and lung and bronchial cancers, metastatic colorectal cancer, metastatic melanoma with CDK4 mutation or amplification, or cisplatin-refractory, unresectable germ cell tumors.

In some embodiments, the Rb-positive cancer is selected from an Rb-positive carcinoma or sarcoma, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

In some embodiments, the Rb-positive cancer is selected from the group consisting of Rb-positive: fibrosarcoma, myxosarcoma, chondrosarcoma, osteosarcoma, chordoma, malignant fibrous histiocytoma, hemangiosarcoma, angiosarcoma, lymphangiosarcoma, Mesothelioma, leiomyosarcoma, rhabdomyosarcoma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phylloide, salivary cancers, thymic carcinomas, bladder cancer, and Wilms tumor.

In more embodiments, the Rb-positive cancer or disorder includes a blood disorder or a hematologic malignancy, including, but not limited to, myeloid disorder, lymphoid disorder, leukemia, lymphoma, myelodysplastic syndrome (MDS), myeloproliferative disease (MPD), mast cell disorder, and myeloma (e.g., multiple myeloma), among others. Abnormal proliferation of T-cells, B-cells, and/or NK-cells can result in a wide range of diseases such as cancer, proliferative disorders and inflammatory/immune diseases. A host, for example a human, afflicted with any of these disorders can be treated with an effective amount of a combination as described herein to achieve a decrease in symptoms (a palliative agent) or a decrease in the underlying disease (a disease modifying agent).

Examples include T-cell or NK-cell lymphoma, for example, but not limited to: peripheral T-cell lymphoma; anaplastic large cell lymphoma, for example anaplastic lymphoma kinase (ALK) positive, ALK negative anaplastic large cell lymphoma, or primary cutaneous anaplastic large cell lymphoma; angioimmunoblastic lymphoma; cutaneous T-cell lymphoma, for example mycosis fungoides, Sézary syndrome, primary cutaneous anaplastic large cell lymphoma, primary cutaneous CD30+ T-cell lymphoproliferative disorder; primary cutaneous aggressive epidermotropic CD8+ cytotoxic T-cell lymphoma; primary cutaneous gamma-delta T-cell lymphoma; primary cutaneous small/medium CD4+ T-cell lymphoma, and lymphomatoid papulosis; Adult T-cell Leukemia/Lymphoma (ATLL); Blastic NK-cell Lymphoma; Enteropathy-type T-cell lymphoma; Hematosplenic gamma-delta T-cell Lymphoma; Lymphoblastic Lymphoma; Nasal NK/T-cell Lymphomas; Treatment-related T-cell lymphomas; for example lymphomas that appear after solid organ or bone marrow transplantation; T-cell prolymphocytic leukemia; T-cell large granular lymphocytic leukemia; Chronic lymphoproliferative disorder of NK-cells; Aggressive NK cell leukemia; Systemic EBV+T-cell lymphoproliferative disease of childhood (associated with chronic active EBV infection); Hydroa vacciniforme-like lymphoma; Adult T-cell leukemia/ lymphoma; Enteropathy-associated T-cell lymphoma; Hepatosplenic T-cell lymphoma; or Subcutaneous panniculitis-like T-cell lymphoma.

In some embodiments, the methods described herein can be used to treat a host, for example a human, with a lymphoma or lymphocytic or myelocytic proliferation disorder or abnormality. For example, the methods as described herein can be administered to a host with a Hodgkin Lymphoma or a Non-Hodgkin Lymphoma. For example, the host can have a Non-Hodgkin Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia.

Alternatively, the methods described herein can be used to treat a subject, for example a human, with a Hodgkin Lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin Lymphoma; or Nodular Lymphocyte Predominant HL.

Alternatively, the methods described herein, can be used to treat a specific B-cell lymphoma or proliferative disorder such as, but not limited to: multiple myeloma; Diffuse large B cell lymphoma; Follicular lymphoma; Mucosa-Associated Lymphatic Tissue lymphoma (MALT); Small cell lymphocytic lymphoma; Mediastinal large B cell lymphoma; Nodal marginal zone B cell lymphoma (NMZL); Splenic marginal zone lymphoma (SMZL); Intravascular large B-cell lymphoma; Primary effusion lymphoma; or Lymphomatoid granulomatosis; B-cell prolymphocytic leukemia; Hairy cell leukemia; Splenic lymphoma/leukemia, unclassifiable; Splenic diffuse red pulp small B-cell lymphoma; Hairy cell leukemia-variant; Lymphoplasmacytic lymphoma; Heavy chain diseases, for example, Alpha heavy chain disease, Gamma heavy chain disease, Mu heavy chain disease; Plasma cell myeloma; Solitary plasmacytoma of bone; Extraosseous plasmacytoma; Primary cutaneous follicle center lymphoma; T cell/histiocyte rich large B-cell lymphoma; DLBCL associated with chronic inflammation; Epstein-Barr virus (EBV)+DLBCL of the elderly; Primary mediastinal (thymic) large B-cell lymphoma; Primary cutaneous DLBCL, leg type; ALK+ large B-cell lymphoma; Plasmablastic lymphoma; Large B-cell lymphoma arising in HHV8-associated multicentric; Castleman disease; B-cell lymphoma, unclassifiable, with features intermediate between diffuse large B-cell lymphoma; or B-cell lymphoma, unclassifiable, with features intermediate between diffuse large B-cell lymphoma and classical Hodgkin lymphoma.

In some embodiments, the methods described herein can be used to treat a leukemia. For example, the subject may be suffering from an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AML); large granular lymphocytic leukemia; or Adult T-cell chronic leukemia. In some embodiments, the patient has an acute myelogenous leukemia, for example an undifferentiated AML (MO); myeloblastic leukemia (Ml; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7).

CDK4/6-replication independent cancers can be deduced based on tumor type and molecular genetics using standard techniques, and can be characterized by one or more of the group including, but not limited to, increased activity of CDK1 or CDK2, loss, deficiency, or absence of retinoblastoma tumor suppressor protein (Rb), high levels of MYC expression, increased cyclin E (e.g., E1 or E2) and increased cyclin A, or expression of a Rb-inactivating protein (such as HPV-encoded E7). Such cancers can include, but are not limited to, small cell lung cancer, retinoblastoma, HPV positive malignancies like cervical cancer and certain head and neck cancers, MYC amplified tumors such as Burkitts' Lymphoma, and triple negative breast cancer; certain classes of sarcoma, certain classes of non-small cell lung carcinoma, certain classes of melanoma, certain classes of pancreatic cancer, certain classes of leukemia, certain classes of lymphoma, certain classes of brain cancer, certain classes of colon cancer, certain classes of prostate cancer, certain classes of ovarian cancer, certain classes of uterine cancer, certain classes of thyroid and other endocrine tissue cancers, certain classes of salivary cancers, certain classes of thymic carcinomas, certain classes of kidney cancers, certain classes of bladder cancers, and certain classes of testicular cancers.

The presence or absence of the retinoblastoma (Rb) tumor suppressor protein (Rb-positive) can be determined through any of the standard assays known to one of ordinary skill in the art, including but not limited to Western Blot, ELISA (enzyme linked immunoadsorbent assay), IHC (immunohistochemistry), and FACS (fluorescent activated cell sorting). The selection of the assay will depend upon the tissue, cell line or surrogate tissue sample that is utilized e.g., for example Western Blot and ELISA may be used with any or all types of tissues, cell lines or surrogate tissues, whereas the IHC method would be more appropriate wherein the tissue utilized in the methods of the present invention was a tumor biopsy. FACs analysis would be most applicable to samples that were single cell suspensions such as cell lines and isolated peripheral blood mononuclear cells. See for example, US 20070212736 “Functional Immunohistochemical Cell Cycle Analysis as a Prognostic Indicator for Cancer”. Alternatively, molecular genetic testing may be used for determination of retinoblastoma gene status. Molecular genetic testing for retinoblastoma includes the following as described in Lohmann and Gallie “Retinoblastoma. Gene Reviews” (2010): “A comprehensive, sensitive and economical approach for the detection of mutations in the RB1 gene in retinoblastoma” Journal of Genetics, 88(4), 517-527 (2009).

### Therapeutic Regimens

In some aspects, methods are provided for the treatment of a subject, typically a human, having cancer comprising administering to the subject a CDK4/6 inhibitor in combination with eribulin, or a pharmaceutically acceptable salt, for example eribulin mesylate, wherein the CDK4/6 inhibitor is administered to the subject about 24 hours or less prior to administration of the eribulin. In certain embodiments, the CDK4/6 inhibitor is selected from Compound 1 and Compound 2. In some embodiments, Compound 1 or Compound 2 is administered about 4 hours or less, for example about 30 minutes or less prior to administration of eribulin or its pharmaceutically acceptable salt.

In some aspects, methods are provided for the preservation of HSPCs in a subject, typically a human, who has an CDK4/6-replication dependent cancer or tumor and is currently undergoing chemotherapy with eribulin or its pharmaceutically acceptable salt comprising administering an effective amount of a selective CDK4/6 inhibitor described herein to the subject in combination with eribulin or its pharmaceutically acceptable salt, wherein the CDK4/6 inhibitor is administered to the subject about 24 hours or less prior to eribulin. In other aspects, methods are provided for the treatment of an Rb-negative cancer or tumor in a subject, typically a human, comprising administering an effective amount of a selective CDK4/6 inhibitor described herein to the subject in combination with eribulin or its pharmaceutically acceptable salt, wherein the CDK4/6 inhibitor is administered to the subject about 24 hours or less prior to administration of eribulin. In certain embodiments, the CDK4/6 inhibitor is selected from Compound 1 and Compound 2. In some embodiments, Compound 1 or Compound 2 is administered about 4 hours or less, for example about 30 minutes or less prior to administration of eribulin or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In some aspects, methods are provided for the prevention or reduction of myelosuppression or myeloablation in a subject, typically a human, who has an CDK4/6-replication dependent cancer or tumor and is currently undergoing chemotherapy with eribulin or its pharmaceutically acceptable salt comprising administering an effective amount of a selective CDK4/6 inhibitor described herein to the subject in combination with eribulin or its pharmaceutically acceptable salt, wherein the CDK4/6 inhibitor is administered to the subject about 24 hours or less prior to eribulin.

In certain embodiments, the CDK4/6 inhibitor is selected from Compound 1 and Compound 2. In some embodiments, Compound 1 or Compound 2 is administered about 4 hours or less, for example about 30 minutes or less prior to administration of eribulin or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In some embodiments, a method is provided for the preservation of HSPCs, or for the prevention or reduction of myelosuppression or myeloablation, in a subject, typically a human, who has metastatic breast cancer and is currently undergoing chemotherapy with eribulin or its pharmaceutically acceptable salt comprising administering an effective amount of a selective CDK4/6 inhibitor to the subject in combination with eribulin or its pharmaceutically acceptable salt, wherein the CDK4/6 inhibitor is administered about 24 hours or less prior to administration of eribulin. In some embodiments, the selective CDK4/6 inhibitor is compound 1 or its pharmaceutically acceptable salt. In another embodiment, the selective CDK4/6 inhibitor is compound 2 or its pharmaceutically acceptable salt. In another embodiment, eribulin is administered on day 1 and day 8 of a 21-day treatment cycle. In another embodiment, eribulin is administered on day 1, day 8 and day 15 of a 28-day treatment cycle. In another embodiment, the selective CDK4/6 inhibitor is administered less than about 2 hours prior to administration of eribulin of its pharmaceutically acceptable salt. In another embodiment, the selective CDK4/6 inhibitor is administered less than about 1 hour prior to administration of eribulin or its pharmaceutically acceptable salt. In another embodiment, the selective CDK4/6 inhibitor is administered about 30 minutes prior to administration of eribulin or its pharmaceutically acceptable salt. In some embodiments, the subject has previously received at least two chemotherapeutic regimens for the treatment of metastatic disease comprising an anthracycline drug and a taxane drug. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin. In certain embodiments, methods of the present invention can also include the administration of one or more additional therapeutic agents. In some embodiments, the additional therapeutic agent is an anti-hormonal agent, for example a SERM (selective estrogen receptor modulator), a SERD (selective estrogen receptor degrader), a complete estrogen receptor degrader, or another form of partial or complete estrogen antagonist, selective androgen receptor modulator, a selective androgen receptor degrader, a complete androgen receptor degrader, or another form of partial or complete androgen antagonist.

In some embodiments, the additional anti-hormonal agent is selected from fulvestrant, tamoxifen, anastrozole, letrozole, exemestane, goserelin, leuprolide, megestrol acetate and toremifene.

In another embodiment, a method is provided for the treatment of metastatic breast cancer in a subject comprising: administering eribulin or its pharmaceutically acceptable salt on day 1 and day 8 of a 21-day treatment cycle; and administering a selective CDK4/6 inhibitor on day 1 and day 8 of a 21-day treatment cycle; wherein the selective CDK4/6 inhibitor is administered about 4 hours or less, for example 30 minutes, prior to administration of eribulin of its pharmaceutically acceptable salt. In another embodiment, a method is provided for the treatment of metastatic breast cancer in a subject comprising: administering eribulin or its pharmaceutically acceptable salt on day 1, day 8 and day 15 of a 28-day treatment cycle; and administering a selective CDK4/6 inhibitor on day 1, day 8 and day 15 of a 28-day treatment cycle; wherein the selective CDK4/6 inhibitor is administered about 4 hours or less, for example 30 minutes, prior to administration of eribulin of its pharmaceutically acceptable salt. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, the subject has previously received at least two chemotherapeutic regimens for the treatment of metastatic disease comprising an anthracycline drug and a taxane drug. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin. In certain embodiments, methods of the present invention can also include the administration of one or more additional therapeutic agents. In some embodiments, the additional therapeutic agent is an anti-hormonal agent, for example a SERM (selective estrogen receptor modulator), a SERD (selective estrogen receptor degrader), a complete estrogen receptor degrader, or another form of partial or complete estrogen antagonist, selective androgen receptor modulator, a selective androgen receptor degrader, a complete androgen receptor degrader, or another form of partial or complete androgen antagonist. In some embodiments, the additional anti-hormonal agent is selected from fulvestrant, tamoxifen, anastrozole, letrozole, exemestane, goserelin, leuprolide, megestrol acetate and toremifene.

In some embodiments, a method is provided for the preservation of HSPCs, or for the prevention or reduction of myelosuppression or myeloablation, in a subject, typically a human, who has breast cancer and is currently undergoing chemotherapy with eribulin or its pharmaceutically acceptable salt comprising administering an effective amount of a selective CDK4/6 inhibitor to the subject in combination with eribulin or its pharmaceutically acceptable salt, wherein the CDK4/6 inhibitor is administered about 24 hours or less prior to administration of eribulin. In certain embodiments, the cancer is a breast cancer selected from the group consisting of triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor negative breast cancer, ductal carcinoma in situ (OCiS), invasive ductal carcinoma, invasive lobular carcinoma, inflammatory breast cancer, Paget disease of the nipple, phyllodes tumor, and a hormone responsive cancer (e.g., hormone responsive breast cancer). In some embodiments, the selective CDK4/6 inhibitor is compound 1 or its pharmaceutically acceptable salt. In another embodiment, the selective CDK4/6 inhibitor is compound 2 or its pharmaceutically acceptable salt. In another embodiment, eribulin is administered on day 1 and day 8 of a 21-day treatment cycle. In another embodiment, eribulin is administered on day 1, day 8 and day 15 of a 28-day treatment cycle. In another embodiment, the selective CDK4/6 inhibitor is administered less than about 2 hours prior to administration of eribulin of its pharmaceutically acceptable salt. In another embodiment, the selective CDK4/6 inhibitor is administered less than about 1 hour prior to administration of eribulin or its pharmaceutically acceptable salt. In another embodiment, the selective CDK4/6 inhibitor is administered about 30 minutes prior to administration of eribulin or its pharmaceutically acceptable salt. In some embodiments, the subject has previously received at least two chemotherapeutic regimens for the treatment of disease comprising an anthracycline drug and a taxane drug. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin. In certain embodiments, methods of the present invention can also include the administration of one or more additional therapeutic agents. In some embodiments, the additional therapeutic agent is an anti-hormonal agent, for example a SERM (selective estrogen receptor modulator), a SERD (selective estrogen receptor degrader), a complete estrogen receptor degrader, or another form of partial or complete estrogen antagonist, selective androgen receptor modulator, a selective androgen receptor degrader, a complete androgen receptor degrader, or another form of partial or complete androgen antagonist. In some embodiments, the additional anti-hormonal agent is selected from fulvestrant, tamoxifen, anastrozole, letrozole, exemestane, goserelin, leuprolide, megestrol acetate and toremifene.

In another embodiment, a method is provided for the treatment of breast cancer in a subject comprising: administering eribulin or its pharmaceutically acceptable salt on day 1 and day 8 of a 21-day treatment cycle; and administering a selective CDK4/6 inhibitor on day 1 and day 8 of a 21-day treatment cycle; wherein the selective CDK4/6 inhibitor is administered about 4 hours or less, for example 30 minutes, prior to administration of eribulin of its pharmaceutically acceptable salt. In another embodiment, a method is provided for the treatment of breast cancer in a subject comprising: administering eribulin or its pharmaceutically acceptable salt on day 1, day 8 and day 15 of a 28-day treatment cycle; and administering a selective CDK4/6 inhibitor on day 1, day 8 and day 15 of a 28-day treatment cycle; wherein the selective CDK4/6 inhibitor is administered about 4 hours or less, for example 30 minutes, prior to administration of eribulin of its pharmaceutically acceptable salt. In certain embodiments, the cancer is a breast cancer selected from the group consisting of triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor negative breast cancer, ductal carcinoma in situ (OCiS), invasive ductal carcinoma, invasive lobular carcinoma, inflammatory breast cancer, Paget disease of the nipple, phyllodes tumor, and a hormone responsive cancer (e.g., hormone responsive breast cancer). In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, the subject has previously received at least two chemotherapeutic regimens for the treatment of disease comprising an anthracycline drug and a taxane drug. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin. In certain embodiments, methods of the present invention can also include the administration of one or more additional therapeutic agents. In some embodiments, the additional therapeutic agent is an anti-hormonal agent, for example a SERM (selective estrogen receptor modulator), a SERD (selective estrogen receptor degrader), a complete estrogen receptor degrader, or another form of partial or complete estrogen antagonist, selective androgen receptor modulator, a selective androgen receptor degrader, a complete androgen receptor degrader, or another form of partial or complete androgen antagonist. In some embodiments, the additional anti-hormonal agent is selected from fulvestrant, tamoxifen, anastrozole, letrozole, exemestane, goserelin, leuprolide, megestrol acetate and toremifene.

In some embodiments, a method is provided for the preservation of HSPCs, or for the prevention or reduction of myelosuppression or myeloablation, in a subject, typically a human, who has unresectable or metastatic liposarcoma and is currently undergoing chemotherapy with eribulin or its pharmaceutically acceptable salt comprising administering an effective amount of a selective CDK4/6 inhibitor to the subject in combination with eribulin or its pharmaceutically acceptable salt, wherein the CDK4/6 inhibitor is administered about 24 hours or less prior to administration of eribulin. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, Compound 1 or Compound 2 is administered about 4 hours or less, for example about 30 minutes or less prior to administration of eribulin or its pharmaceutically acceptable salt. In another embodiment, eribulin is administered on day 1 and day 8 of a 21-day treatment cycle. In another embodiment, eribulin is administered on day 1, day 8 and day 15 of a 28-day treatment cycle. In another embodiment, the selective CDK4/6 inhibitor is administered less than about 2 hours prior to administration of eribulin of its pharmaceutically acceptable salt. In another embodiment, the selective CDK4/6 inhibitor is administered less than about 1 hour prior to administration of eribulin or its pharmaceutically acceptable salt. In another embodiment, the selective CDK4/6 inhibitor is administered about 30 minutes prior to administration of eribulin or its pharmaceutically acceptable salt. In some embodiments, the subject has previously received at anthracycline-containing regimen. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In another embodiment, a method is provided for the treatment of unresectable or metastatic liposarcoma in a subject comprising: administering eribulin or its pharmaceutically acceptable salt on day 1 and day 8 of a 21-day treatment cycle; and administering a selective CDK4/6 inhibitor on day 1 and day 8 of a 21-day treatment cycle; wherein the selective CDK4/6 inhibitor is administered about 30 minutes prior to administration of eribulin of its pharmaceutically acceptable salt. In another embodiment, a method is provided for the treatment of unresectable or metastatic liposarcoma in a subject comprising: administering eribulin or its pharmaceutically acceptable salt on day 1, day 8 and day 15 of a 28-day treatment cycle; and administering a selective CDK4/6 inhibitor on day 1, day 8 and day 15 of a 28-day treatment cycle; wherein the selective CDK4/6 inhibitor is administered about 30 minutes prior to administration of eribulin of its pharmaceutically acceptable salt. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, the subject has previously received at anthracycline-containing regimen. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In some embodiments, a method is provided for the preservation of HSPCs, or for the prevention or reduction of myelosuppression or myeloablation, in a subject, typically a human, who has relapsed or refractory rhabdomyosarcoma and is currently undergoing chemotherapy with eribulin or its pharmaceutically acceptable salt comprising administering an effective amount of a selective CDK4/6 inhibitor to the subject in combination with eribulin or its pharmaceutically acceptable salt, wherein the CDK4/6 inhibitor is administered about 24 hours or less prior to administration of eribulin. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In another embodiment, eribulin is administered on day 1 and day 8 of a 21-day treatment cycle. In another embodiment, eribulin is administered on day 1, day 8 and day 15 of a 28-day treatment cycle. In another embodiment, the selective CDK4/6 inhibitor is administered less than about 2 hours prior to administration of eribulin of its pharmaceutically acceptable salt. In another embodiment, the selective CDK4/6 inhibitor is administered less than about 1 hour prior to administration of eribulin or its pharmaceutically acceptable salt. In another embodiment, the selective CDK4/6 inhibitor is administered about 30 minutes prior to administration of eribulin or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In another embodiment, a method is provided for the treatment of relapsed or refractory rhabdomyosarcoma in a subject comprising: administering eribulin or its pharmaceutically acceptable salt on day 1 and day 8 of a 21-day treatment cycle; and administering a selective CDK4/6 inhibitor on day 1 and day 8 of a 21-day treatment cycle; wherein the selective CDK4/6 inhibitor is administered about 30 minutes prior to administration of eribulin of its pharmaceutically acceptable salt. In another embodiment, a method is provided for the treatment of relapsed or refractory rhabdomyosarcoma in a subject comprising: administering eribulin or its pharmaceutically acceptable salt on day 1, day 8 and day 15 of a 28-day treatment cycle; and administering a selective CDK4/6 inhibitor on day 1, day 8 and day 15 of a 28-day treatment cycle; wherein the selective CDK4/6 inhibitor is administered about 30 minutes prior to administration of eribulin of its pharmaceutically acceptable salt. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In some embodiments, a method is provided for the preservation of HSPCs, or for the prevention or reduction of myelosuppression or myeloablation, in a subject, typically a human, who has non-rhabdomyosarcoma soft tissue sarcoma and is currently undergoing chemotherapy with eribulin or its pharmaceutically acceptable salt comprising administering an effective amount of a selective CDK4/6 inhibitor to the subject in combination with eribulin or its pharmaceutically acceptable salt, wherein the CDK4/6 inhibitor is administered about 24 hours or less prior to administration of eribulin. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, Compound 1 or Compound 2 is administered about 4 hours or less, for example about 30 minutes or less prior to administration of eribulin or its pharmaceutically acceptable salt. In another embodiment, eribulin is administered on day 1 and day 8 of a 21-day treatment cycle. In another embodiment, eribulin is administered on day 1, day 8 and day 15 of a 28-day treatment cycle. In another embodiment, the selective CDK4/6 inhibitor is administered less than about 2 hours prior to administration of eribulin of its pharmaceutically acceptable salt. In another embodiment, the selective CDK4/6 inhibitor is administered less than about 1 hour prior to administration of eribulin or its pharmaceutically acceptable salt. In another embodiment, the selective CDK4/6 inhibitor is administered about 30 minutes prior to administration of eribulin or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In another embodiment, a method is provided for the treatment of non-rhabdomyosarcoma soft tissue sarcoma in a subject comprising: administering eribulin or its pharmaceutically acceptable salt on day 1 and day 8 of a 21-day treatment cycle; and administering a selective CDK4/6 inhibitor on day 1 and day 8 of a 21-day treatment cycle; wherein the selective CDK4/6 inhibitor is administered about 30 minutes prior to administration of eribulin of its pharmaceutically acceptable salt. In another embodiment, a method is provided for the treatment of non-rhabdomyosarcoma soft tissue sarcoma in a subject comprising: administering eribulin or its pharmaceutically acceptable salt on day 1, day 8 and day 15 of a 28-day treatment cycle; and administering a selective CDK4/6 inhibitor on day 1, day 8 and day 15 of a 28-day treatment cycle; wherein the selective CDK4/6 inhibitor is administered about 30 minutes prior to administration of eribulin of its pharmaceutically acceptable salt. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In some embodiments, a method is provided for the preservation of HSPCs, or for the prevention or reduction of myelosuppression or myeloablation, in a subject, typically a human, who has Ewing sarcoma and is currently undergoing chemotherapy with eribulin or its pharmaceutically acceptable salt comprising administering an effective amount of a selective CDK4/6 inhibitor to the subject in combination with eribulin or its pharmaceutically acceptable salt, wherein the CDK4/6 inhibitor is administered about 24 hours or less prior to administration of eribulin. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, Compound 1 or Compound 2 is administered about 4 hours or less, for example about 30 minutes or less prior to administration of eribulin or its pharmaceutically acceptable salt. In another embodiment, eribulin is administered on day 1 and day 8 of a 21-day treatment cycle. In another embodiment, eribulin is administered on day 1, day 8 and day 15 of a 28-day treatment cycle. In another embodiment, the selective CDK4/6 inhibitor is administered less than about 2 hours prior to administration of eribulin of its pharmaceutically acceptable salt. In another embodiment, the selective CDK4/6 inhibitor is administered less than about 1 hour prior to administration of eribulin or its pharmaceutically acceptable salt. In another embodiment, the selective CDK4/6 inhibitor is administered about 30 minutes prior to administration of eribulin or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In another embodiment, a method is provided for the treatment of Ewing sarcoma in a subject comprising: administering eribulin or its pharmaceutically acceptable salt on day 1 and day 8 of a 21-day treatment cycle; and administering a selective CDK4/6 inhibitor on day 1 and day 8 of a 21-day treatment cycle; wherein the selective CDK4/6 inhibitor is administered about 30 minutes prior to administration of eribulin of its pharmaceutically acceptable salt. In another embodiment, a method is provided for the treatment of Ewing sarcoma in a subject comprising: administering eribulin or its pharmaceutically acceptable salt on day 1, day 8 and day 15 of a 28-day treatment cycle; and administering a selective CDK4/6 inhibitor on day 1, day 8 and day 15 of a 28-day treatment cycle; wherein the selective CDK4/6 inhibitor is administered about 30 minutes prior to administration of eribulin of its pharmaceutically acceptable salt. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In some embodiments, a method is provided for the preservation of HSPCs, or for the prevention or reduction of myelosuppression or myeloablation, in a subject, typically a human, who has angiosarcoma and is currently undergoing chemotherapy with eribulin or its pharmaceutically acceptable salt comprising administering an effective amount of a selective CDK4/6 inhibitor to the subject in combination with eribulin or its pharmaceutically acceptable salt, wherein the CDK4/6 inhibitor is administered about 24 hours or less prior to administration of eribulin. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, Compound 1 or Compound 2 is administered about 4 hours or less, for example about 30 minutes or less prior to administration of eribulin or its pharmaceutically acceptable salt. In another embodiment, eribulin is administered on day 1 and day 8 of a 21-day treatment cycle. In another embodiment, eribulin is administered on day 1, day 8 and day 15 of a 28-day treatment cycle. In another embodiment, the selective CDK4/6 inhibitor is administered less than about 2 hours prior to administration of eribulin of its pharmaceutically acceptable salt. In another embodiment, the selective CDK4/6 inhibitor is administered less than about 1 hour prior to administration of eribulin or its pharmaceutically acceptable salt. In another embodiment, the selective CDK4/6 inhibitor is administered about 30 minutes prior to administration of eribulin or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In another embodiment, a method is provided for the treatment angiosarcoma in a subject comprising: administering eribulin or its pharmaceutically acceptable salt on day 1 and day 8 of a 21-day treatment cycle; and administering a selective CDK4/6 inhibitor on day 1 and day 8 of a 21-day treatment cycle; wherein the selective CDK4/6 inhibitor is administered about 30 minutes prior to administration of eribulin of its pharmaceutically acceptable salt. In another embodiment, a method is provided for the treatment angiosarcoma in a subject comprising: administering eribulin or its pharmaceutically acceptable salt on day 1, day 8 and day 15 of a 28-day treatment cycle; and administering a selective CDK4/6 inhibitor on day 1, day 8 and dayl5 of a 28-day treatment cycle; wherein the selective CDK4/6 inhibitor is administered about 30 minutes prior to administration of eribulin of its pharmaceutically acceptable salt. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In some embodiments, a method is provided for the preservation of HSPCs, or for the prevention or reduction of myelosuppression or myeloablation, in a subject, typically a human, who has epithelioid hemangioendothelioma and is currently undergoing chemotherapy with eribulin or its pharmaceutically acceptable salt comprising administering an effective amount of a selective CDK4/6 inhibitor to the subject in combination with eribulin or its pharmaceutically acceptable salt, wherein the CDK4/6 inhibitor is administered about 24 hours or less prior to the administration of eribulin. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, Compound 1 or Compound 2 is administered about 4 hours or less, for example about 30 minutes or less prior to administration of eribulin or its pharmaceutically acceptable salt. In another embodiment, eribulin is administered on day 1 and day 8 of a 21-day treatment cycle. In another embodiment, eribulin is administered on day 1, day 8 and day 15 of a 28-day treatment cycle. In another embodiment, the selective CDK4/6 inhibitor is administered less than about 2 hours prior to administration of eribulin of its pharmaceutically acceptable salt. In another embodiment, the selective CDK4/6 inhibitor is administered less than about 1 hour prior to administration of eribulin or its pharmaceutically acceptable salt. In another embodiment, the selective CDK4/6 inhibitor is administered about 30 minutes prior to administration of eribulin or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In another embodiment, a method is provided for the treatment epithelioid hemangioendothelioma in a subject comprising: administering eribulin or its pharmaceutically acceptable salt on day 1 and day 8 of a 21-day treatment cycle; and administering a selective CDK4/6 inhibitor on day 1 and day 8 of a 21-day treatment cycle; wherein the selective CDK4/6 inhibitor is administered about 30 minutes prior to administration of eribulin of its pharmaceutically acceptable salt. In another embodiment, a method is provided for the treatment epithelioid hemangioendothelioma in a subject comprising: administering eribulin or its pharmaceutically acceptable salt on day 1, day 8 and day 15 of a 28-day treatment cycle; and administering a selective CDK4/6 inhibitor on day 1, day 8 and day 15 of a 28-day treatment cycle; wherein the selective CDK4/6 inhibitor is administered about 30 minutes prior to administration of eribulin of its pharmaceutically acceptable salt. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In some embodiments, a method is provided for the preservation of HSPCs, or for the prevention or reduction of myelosuppression or myeloablation, in a subject, typically a human, who has metastatic urothelial cell cancer and is currently undergoing chemotherapy with eribulin or its pharmaceutically acceptable salt comprising administering an effective amount of a selective CDK4/6 inhibitor to the subject in combination with eribulin or its pharmaceutically acceptable salt, wherein the CDK4/6 inhibitor is administered about 24 hours or less prior to the administration of eribulin. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, Compound 1 or Compound 2 is administered about 4 hours or less, for example about 30 minutes or less prior to administration of eribulin or its pharmaceutically acceptable salt. In another embodiment, eribulin is administered on day 1 and day 8 of a 21-day treatment cycle. In another embodiment, eribulin is administered on day 1, day 8 and day 15 of a 28-day treatment cycle. In another embodiment, the selective CDK4/6 inhibitor is administered less than about 2 hours prior to administration of eribulin of its pharmaceutically acceptable salt. In another embodiment, the selective CDK4/6 inhibitor is administered less than about 1 hour prior to administration of eribulin or its pharmaceutically acceptable salt. In another embodiment, the selective CDK4/6 inhibitor is administered about 30 minutes prior to administration of eribulin or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In another embodiment, a method is provided for the treatment of metastatic urothelial cell cancer in a subject comprising: administering eribulin or its pharmaceutically acceptable salt on day 1 and day 8 of a 21-day treatment cycle; and administering a selective CDK4/6 inhibitor on day 1 and day 8 of a 21-day treatment cycle; wherein the selective CDK4/6 inhibitor is administered about 30 minutes prior to administration of eribulin of its pharmaceutically acceptable salt. In another embodiment, a method is provided for the treatment of metastatic urothelial cell cancer in a subject comprising: administering eribulin or its pharmaceutically acceptable salt on day 1, day 8 and day 15 of a 28-day treatment cycle; and administering a selective CDK4/6 inhibitor on day 1, day 8 and day 15 of a 28-day treatment cycle; wherein the selective CDK4/6 inhibitor is administered about 30 minutes prior to administration of eribulin of its pharmaceutically acceptable salt. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In some embodiments, a method is provided for the preservation of HSPCs, or for the prevention or reduction of myelosuppression or myeloablation, in a subject, typically a human, who has non-small cell lung cancer and is currently undergoing chemotherapy with eribulin or its pharmaceutically acceptable salt comprising administering an effective amount of a selective CDK4/6 inhibitor to the subject in combination with eribulin or its pharmaceutically acceptable salt, wherein the selective CDK4/6 inhibitor is administered about 24 hours or less prior to the administration of eribulin. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, Compound 1 or Compound 2 is administered about 4 hours or less, for example about 30 minutes or less prior to administration of eribulin or its pharmaceutically acceptable salt. In another embodiment, eribulin is administered on day 1 and day 8 of a 21-day treatment cycle. In another embodiment, eribulin is administered on day 1, day 8 and day 15 of a 28-day treatment cycle. In another embodiment, the selective CDK4/6 inhibitor is administered less than about 2 hours prior to administration of eribulin of its pharmaceutically acceptable salt. In another embodiment, the selective CDK4/6 inhibitor is administered less than about 1 hour prior to administration of eribulin or its pharmaceutically acceptable salt. In another embodiment, the selective CDK4/6 inhibitor is administered about 30 minutes prior to administration of eribulin or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In another embodiment, a method is provided for the treatment of non-small cell lung cancer in a subject comprising: administering eribulin or its pharmaceutically acceptable salt on day 1 and day 8 of a 21-day treatment cycle; and administering a selective CDK4/6 inhibitor on day 1 and day 8 of a 21-day treatment cycle; wherein the selective CDK4/6 inhibitor is administered about 30 minutes prior to administration of eribulin of its pharmaceutically acceptable salt. In another embodiment, a method is provided for the treatment of non-small cell lung cancer in a subject comprising: administering eribulin or its pharmaceutically acceptable salt on day 1, day 8 and day 15 of a 28-day treatment cycle; and administering a selective CDK4/6 inhibitor on day 1, day 8 and day 15 of a 28-day treatment cycle; wherein the selective CDK4/6 inhibitor is administered about 30 minutes prior to administration of eribulin of its pharmaceutically acceptable salt. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In some embodiments, a method is provided for the preservation of HSPCs, or for the prevention or reduction of myelosuppression or myeloablation, in a subject, typically a human, who has prostate cancer and is currently undergoing chemotherapy with eribulin or its pharmaceutically acceptable salt comprising administering an effective amount of a selective CDK4/6 inhibitor to the subject in combination with eribulin or its pharmaceutically acceptable salt, wherein the CDK4/6 inhibitor is administered about 24 hours or less prior to the administration of eribulin. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, Compound 1 or Compound 2 is administered about 4 hours or less, for example about 30 minutes or less prior to administration of eribulin or its pharmaceutically acceptable salt. In another embodiment, eribulin is administered on day 1 and day 8 of a 21-day treatment cycle. In another embodiment, eribulin is administered on day 1, day 8 and day 15 of a 28-day treatment cycle. In another embodiment, the selective CDK4/6 inhibitor is administered less than about 2 hours prior to administration of eribulin of its pharmaceutically acceptable salt. In another embodiment, the selective CDK4/6 inhibitor is administered less than about 1 hour prior to administration of eribulin or its pharmaceutically acceptable salt. In another embodiment, the selective CDK4/6 inhibitor is administered about 30 minutes prior to administration of eribulin or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In another embodiment, a method is provided for the treatment of prostate cancer in a subject comprising: administering eribulin or its pharmaceutically acceptable salt on day 1 and day 8 of a 21-day treatment cycle; and administering a selective CDK4/6 inhibitor on day 1 and day 8 of a 21-day treatment cycle; wherein the selective CDK4/6 inhibitor is administered about 30 minutes prior to administration of eribulin of its pharmaceutically acceptable salt. In another embodiment, a method is provided for the treatment of prostate cancer in a subject comprising: administering eribulin or its pharmaceutically acceptable salt on day 1, day 8 and day 15 of a 28-day treatment cycle; and administering a selective CDK4/6 inhibitor on day 1, day 8 and day 15 of a 28-day treatment cycle; wherein the selective CDK4/6 inhibitor is administered about 30 minutes prior to administration of eribulin of its pharmaceutically acceptable salt. In some embodiments, the selective CDK4/6 inhibitor is Compound 1 or its pharmaceutically acceptable salt. In another embodiment, the selective CDK4/6 inhibitor is Compound 2 or its pharmaceutically acceptable salt. In some embodiments, the CDK4/6 inhibitor is administered at two time points prior to administration of eribulin. In some embodiments, the CDK4/6 inhibitor is administered about 24 hours prior to administration of eribulin and again about 4 hours or less, for example, about 30 minutes prior to administration of eribulin.

In an alternative embodiment, a method is provided for the treatment of a cancer or tumor in a subject, typically a human, comprising administering a selective CDK4/6 inhibitor described herein in combination with eribulin or its pharmaceutically acceptable salt, wherein the CDK4/6 inhibitor is administered at least once a day on days 1-21 of a 21-day treatment cycle, and wherein the subject is administered eribulin or its pharmaceutically acceptable salt, e.g., eribulin mesylate, on day 1 and day 8 of a 21-day treatment cycle. In some embodiments, the CDK4/6 inhibitor is Compound 1, or a pharmaceutically acceptable salt composition, isotopic analog, or prodrug thereof. In some embodiments, the selective CDK4/6 inhibitor is Compound 2, or a pharmaceutically acceptable salt, e.g., dihydrochloride salt, composition, isotopic analog, or prodrug thereof. In some embodiments, the cancer or tumor is a CDK4/6-replication dependent cancer or tumor. In some embodiments, the cancer or tumor is a CDK4/6-replication independent cancer or tumor. In some embodiments, the subject has an Rb-positive cancer or tumor, for example, but not limited to metastatic breast cancer, unresectable/metastatic liposarcoma, non-small cell lung cancer, prostate cancer, pancreatic cancer, colorectal cancer, bladder cancer, osteosarcoma, leiomyosarcoma, ovarian cancer, cervical cancer, colon cancer, head and neck cancer, sarcoma, relapsed/refractory rhabdomyosarcoma, non-rhabdomyosarcoma soft tissue sarcoma, Ewing sarcoma, angiosarcoma, epithelioid hemangioendothelioma, and urothelial cell cancer. In some embodiments, the subject is administered Compound 2, or a pharmaceutically acceptable salt thereof, e.g., dihydrochloride salt, concomitantly or prior to, for example less than about 8 hours, less than about 7 hours, less than about 6 hours, less than about 5 hours, less than about 4 hours, less than about 3 hours, less than about 2 hours, less than about 1 hour, or about 30 minutes prior to, administration of eribulin or its pharmaceutically acceptable salt, e.g., eribulin mesylate. In some embodiments, the subject has metastatic breast cancer and has received at least two chemotherapeutic regimens for the treatment of metastatic disease comprising an anthracycline and a taxane drug. In another embodiment, the subject has unresectable or metastatic liposarcoma and has received a prior anthracycline-containing regimen. In certain embodiments, methods of the present invention can also include the administration of one or more additional therapeutic agents. In some embodiments, the additional therapeutic agent is an anti-hormonal agent, for example a SERM (selective estrogen receptor modulator), a SERD (selective estrogen receptor degrader), a complete estrogen receptor degrader, or another form of partial or complete estrogen antagonist, selective androgen receptor modulator, a selective androgen receptor degrader, a complete androgen receptor degrader, or another form of partial or complete androgen antagonist. In some embodiments, the additional anti-hormonal agent is selected from fulvestrant, tamoxifen, anastrozole, letrozole, exemestane, goserelin, leuprolide, megestrol acetate and toremifene.

In an alternative embodiment, a method is provided for the treatment of a cancer or tumor in a subject, typically a human, comprising administering a selective CDK4/6 inhibitor described herein in combination or alternation with eribulin or its pharmaceutically acceptable salt, e.g., eribulin mesylate, wherein the CDK4/6 inhibitor is administered at least once daily on days 1-28 of a 28-day treatment cycle whereas the subject is administered eribulin or its pharmaceutically acceptable salt on day 1, day 8 and day 15 of a 28-day treatment cycle. In some embodiments, the selective CDK4/6 inhibitor is Compound 1. In some embodiments, the selective CDK4/6 inhibitor is Compound 2, or a pharmaceutically acceptable salt, e.g., dihydrochloride salt, composition, isotopic analog, or prodrug thereof. In some embodiments, the cancer or tumor is a CDK4/6-replication dependent cancer or tumor. In some embodiments, the cancer or tumor is a CDK4/6-replication independent cancer or tumor. In some embodiments, the subject has an Rb-positive cancer or tumor, for example metastatic breast cancer, unresectable/metastatic liposarcoma, non-small cell lung cancer, prostate cancer, pancreatic cancer, colorectal cancer, bladder cancer, osteosarcoma, leiomyosarcoma, ovarian cancer, cervical cancer, colon cancer, head and neck cancer, sarcoma, relapsed/refractory rhabdomyosarcoma, non-rhabdomyosarcoma soft tissue sarcoma, Ewing sarcoma, angiosarcoma, epithelioid hemangioendothelioma, and urothelial cell cancer. In some embodiments, the subject is administered Compound 2, or a pharmaceutically acceptable salt thereof, e.g., dihydrochloride salt, concomitantly or prior to, for example less than about 8 hours, less than about 7 hours, less than about 6 hours, less than about 5 hours, less than about 4 hours, less than about 3 hours, less than about 2 hours, less than about 1 hour, or about 30 minutes prior to, administration of eribulin or its pharmaceutically acceptable salt e.g., eribulin mesylate. In some embodiments, the subject has metastatic breast cancer and has received at least two chemotherapeutic regimens for the treatment of metastatic disease comprising an anthracycline and a taxane drug. In another embodiment, the subject has unresectable or metastatic liposarcoma and has received a prior anthracycline-containing regimen. In certain embodiments, methods of the present invention can also include the administration of one or more additional therapeutic agents. In some embodiments, the additional therapeutic agent is an anti-hormonal agent, for example a SERM (selective estrogen receptor modulator), a SERD (selective estrogen receptor degrader), a complete estrogen receptor degrader, or another form of partial or complete estrogen antagonist, selective androgen receptor modulator, a selective androgen receptor degrader, a complete androgen receptor degrader, or another form of partial or complete androgen antagonist. In some embodiments, the additional anti-hormonal agent is selected from fulvestrant, tamoxifen, anastrozole, letrozole, exemestane, goserelin, leuprolide, megestrol acetate and toremifene.

### EXPERIMENTAL EXAMPLES OF THE PRESENT INVENTION

### Example 1

### Compound 1 Does Not Decrease Eribulin Efficacy in CDK4/6-Dependent Cell-Based Xenograft Cancer Models

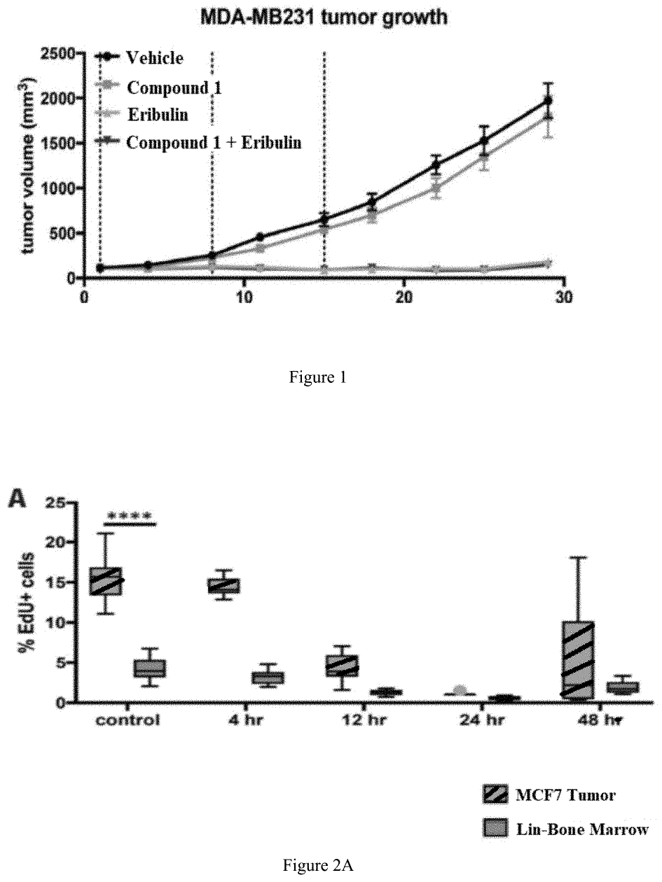

A highly CDK4/6 dependent breast cancer xenograft model (MDA-MB-231) was employed to determine whether transient CDK4/6 inhibition with Compound 1 would antagonize the intended therapeutic effects. First, MD-MB-231 tumor-bearing mice were treated daily with Compound 1 (IP, 100 mg/kg, n=6-8) for 28 days to confirm the CDK4/6 dependency of the xenograft model. Next, MDA-MB-231 tumor-bearing mice were treated with eribulin (IV, 0.5 mg/kg) with or without Compound 1 (IP, 100 mg/kg) weekly for three weeks, with Compound 1 being given 30 minutes prior to chemotherapy treatment. In all experiments, tumors were measured, and tumor volume was calculated twice weekly. As shown in FIG. 1, administration of Compound 1 had no antagonist effect on eribulin therapy when compared to eribulin therapy alone. Therefore, Compound 1 can be administered to preserve hematopoietic stem and progenitor cell (HSPC) and immune system function during eribulin therapy without antagonizing the intended anti-tumor efficacy of eribulin. Compound 1 alone did not demonstrate any significant anti-tumor effect when dosed on a similar schedule as when used in combination with eribulin.

### Example 2

### Comparison of Cell Cycle Kinetics of Bone Marrow and MCF7 Tumor Cells

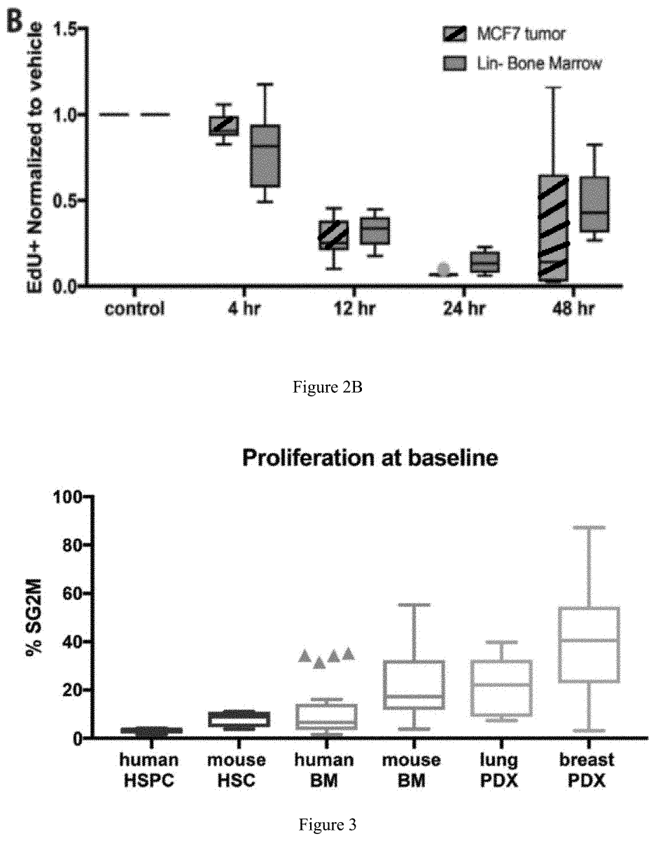

MCF7 tumor-bearing mice were treated with a single dose of Compound 1 (IP, 100 mg/kg) or vehicle control. After 4, 12, 24, and 48 hours of treatment, animals were pulsed with 5-ethynyl-2′-deoxyuridine (EdU; IP, 200 □g). Tumors and femurs from each animal were harvested after 4 hours of EdU dosing and processed to single cell suspensions for detection of EdU+ cells by flow cytometry. HSPC in bone marrow is defined as cell populations negative for lineage markers (Mac-1, Gr-1, Ter119, B220, CD4, CD8). As shown in FIG. 2A, the mean percentage of cycling MCF7 tumor cells at baseline (15.57%) is significantly higher than the percentage of cycling cells in lineage negative (Lin−) bone marrow (4.1%) as measured by EdU incorporation (p=1.37e-13). As shown in FIG. 2B, when normalized to baseline, maximal cell cycle inhibition in both cell types is observed 24 hours post Compound 1 treatment with both cell types reentering the cell cycle by 48 hours post treatment. Students T tests were completed to compare % EdU+ cells (tumor vs bone marrow) at baseline in panel A ****p≤0.0001.

Direct comparison of cell cycle kinetics of bone marrow and MCF7 tumor cells following Compound 1 administration demonstrates that there is a significantly higher fraction of proliferating tumor cells compared to bone marrow at baseline through 24 hours. Chemotherapy treatment is effective at killing cells in the S/G2/M phases of the cell cycle. Compound 1 maintains nearly universal G1-arrest of hematopoietic stem cells while a significant fraction of tumor cells are past the G1 checkpoint (in S/G2/M; as shown in FIGS. 2A and 2B), thereby creating a therapeutic window for the selective protection of bone marrow versus CDK4/6-dependent tumor cells from the cytotoxic effects of chemotherapy, including eribulin.

### Example 3

Comparison of Cell Cycle Kinetics of Bone Marrow vs. MCF7 Tumor Cells After Compound 1 Treatment

To further evaluate the difference in cell cycle kinetics between bone marrow and tumor cells as an explanation for why Compound 1 does not antagonize chemotherapy efficacy in CDK4/6-dependent tumor models, the differences in baseline proliferation rates of hematopoietic stem and progenitor (HSPCs), total bone marrow, and PDX tumors cells were examined using flow cytometric analysis of the cell cycle. The bar graph in FIG. 3 depicts mean percentage of cells in S/G2/M phase of the cell cycle.

As shown in FIG. 3, there are a higher proportion of cycling PDX tumor cells (cells in S/G2/M) when compared to total bone marrow or the HSPC compartment from both mice and humans. These findings are likely to translate to the clinic as: PDX models have been shown to more faithfully replicate the human disease (Dobrolecki et al. 2016). The lowest baseline proliferation was seen in human bone marrow and HSPCs suggesting that the therapeutic window observed from differences in the fraction of proliferating cells may be even larger in patients.

### Example 4

### Daily Dosing of Compound 1 Does Not Decrease Chemotherapy Efficacy in CDK4/6-Dependent Cell-Based Xenograft Cancer Models

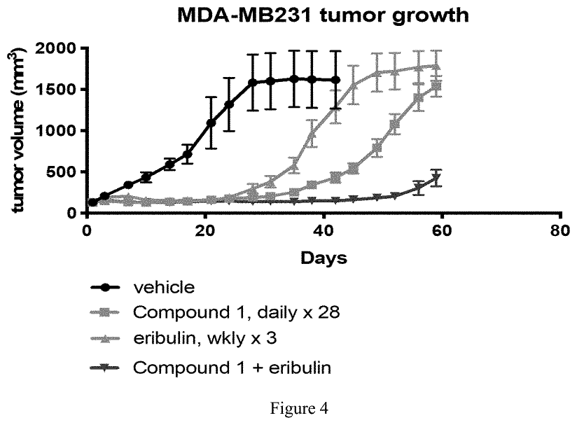

A highly CDK4/6 dependent breast cancer xenograft model (MDA-MB-231) was employed to determine whether continuous CDK4/6 inhibition with Compound 1 would antagonize the intended therapeutic effects of eribulin. First, MDA-MB-231 tumor-bearing mice were treated daily with Compound 1 (IP, 100 mg/kg, n=10) for 28 days to confirm the CDK4/6 dependency of the xenograft model. Next, MDA-MB-231 tumor-bearing mice were treated with eribulin (IV, 0.5 mg/kg, n=10) weekly for three weeks. Next, MDA-MB-231 tumor-bearing mice were treated daily with Compound 1 (IP, 100 mg/kg, n=10) and with eribulin (IV, 0.5 mg/kg, n=10) weekly for three weeks, with Compound 1 being given 30 minutes prior to chemotherapy treatment on days 1, 8, and 15. In all experiments, tumors were measured, and tumor volume was calculated twice weekly. The dosing schedule is shown below in Table 1. As shown in FIG. 4, continuous administration of Compound 1 had a synergistic effect on eribulin therapy when compared to eribulin therapy alone. Therefore, Compound 1 can be administered to preserve hematopoietic stem and progenitor cell (HSPC) and immune system function during eribulin therapy without antagonizing the intended anti-tumor efficacy of eribulin. Compound 1 alone did demonstrate a significant anti-tumor effect when dosed on a daily schedule.

TABLE 1

| | | Treatment Regimen 1 | | | | Treatment Regimen 2 | | | |
|---|---|---|---|---|---|---|---|---|---|
| Group | n | Agent | mg/kg | Route | Schedule | Agent | mg/kg | Route | Schedule |
| 1 | 10 | vehicle | — | ip | qwk × 3 | — | — | — | — |
| 2 | 10 | G1T28 | 100 | ip | qd × 28 | — | — | — | — |
| 3 | 10 | eribulin | 0.5 | iv | qwk × 3 | — | — | — | — |
| 4 | 10 | G1T28 | 100 | ip | qd × 28 | eribulin | 0.5 | iv | qwk x 3 |

Table 1 displays the study design as of Day 1 of the study.
vehicle = 50 mM Citrate Buffer pH 4.5

This specification has been described with reference to embodiments of the invention. The invention has been described with reference to assorted embodiments, which are illustrated by the accompanying Examples. The invention can, however, be embodied in different forms and should not be construed as limited to the embodiments set forth herein. Given the teaching herein, one of ordinary skill in the art will be able to modify the invention for a desired purpose, and such variations are considered within the scope of the invention.

What is claimed is:

1. A method for treating cancer in a human comprising:
administrating to the human an effective amount of a selective CDK4/6 inhibitor; and
administering to the human an effective amount of eribulin, or a pharmaceutically acceptable salt thereof;
wherein the CDK4/6 inhibitor is administered 4 hours or less prior to administration of eribulin;
and wherein the selective CDK4/6 inhibitor is 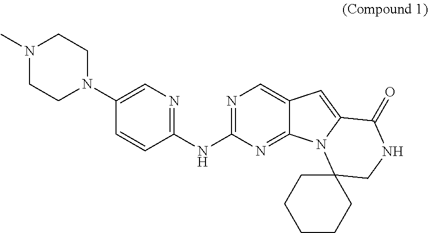 [SMILES: CN1CCN(c2ccc(Nc3ncc4cc5n(c4n3)C3(CCCCC3)CNC5=O)nc2)CC1]
or or a pharmaceutically acceptable salt thereof.

2. The method of claim 1, wherein the pharmaceutically acceptable salt of eribulin is eribulin mesylate.

3. The method of claim 1, wherein the cancer is selected from the group consisting of breast cancer, unresectable/metastatic liposarcoma, non-small cell lung cancer, prostate cancer, pancreatic cancer, colorectal cancer, bladder cancer, osteosarcoma, leiomyosarcoma, ovarian cancer, cervical cancer, colon cancer, head and neck cancer, sarcoma, relapsed/refractory rhabdomyosarcoma, non-rhabdomyosarcoma soft tissue sarcoma, Ewing sarcoma, angiosarcoma, epithelioid hemangioendothelioma, and urothelial cell cancer.

4. The method of claim 3, wherein the breast cancer is selected from the group consisting of metastatic breast cancer, triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor negative breast cancer, ductal carcinoma in situ (OCiS), invasive ductal carcinoma, invasive lobular carcinoma, inflammatory breast cancer, Paget disease of the nipple, phyllodes tumor, and a hormone responsive cancer.

5. The method of claim 1, wherein the cancer is a CDK4/6-replication dependent cancer.

6. The method of claim 1, wherein the cancer is a CDK4/6 replication independent cancer.

7. The method of claim 1, wherein the human is administered the CDK4/6 inhibitor about 30 minutes or less prior to administration of eribulin, or its pharmaceutically acceptable salt.

8. The method of claim 1, wherein the eribulin is administered on days 1 and 8 of a 21-day chemotherapeutic cycle, and the CDK4/6 inhibitor is administered on days 1 and 8 of a 21-day chemotherapeutic cycle.

9. The method of claim 1, wherein the eribulin is administered on days 1, 8, and 15 of a 28-day chemotherapeutic cycle, and the CDK4/6 inhibitor is administered on days 1, 8, and 15 of a 28-day chemotherapeutic cycle.

10. The method of claim 4, further comprising the administration of an anti-hormonal agent, wherein the anti-hormonal agent is selected from the group consisting of a SERM (selective estrogen receptor modulator), a SERD (selective estrogen receptor degrader), a complete estrogen receptor degrader, or another form of partial or complete estrogen antagonist, selective androgen receptor modulator, a selective androgen receptor degrader, a complete androgen receptor degrader, and another form of partial or complete androgen antagonist.

11. The method of claim 10, wherein the anti-hormonal agent is selected from the group consisting of fulvestrant, tamoxifen, anastrozole, letrozole, exemestane, goserelin, and leuprolide.

12. A method for reducing myelosuppression in a human receiving eribulin for the treatment of a cancer comprising:
administrating to the human an effective amount of a selective CDK4/6 inhibitor; and
administering to the human an effective amount of eribulin, or a pharmaceutically acceptable salt thereof;
wherein the CDK4/6 inhibitor is administered 4 hours or less prior to administration of eribulin;
and wherein the selective CDK4/6 inhibitor is 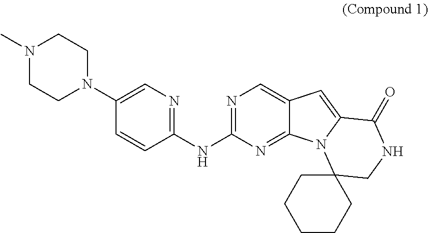 [SMILES: CN1CCN(c2ccc(Nc3ncc4cc5n(c4n3)C3(CCCCC3)CNC5=O)nc2)CC1]
or or a pharmaceutically acceptable salt thereof.

13. The method of claim 12, wherein the pharmaceutically acceptable salt of eribulin is eribulin mesylate.

14. The method of claim 12, wherein the cancer is selected from the group consisting of breast cancer, unresectable/metastatic liposarcoma, non-small cell lung cancer, prostate cancer, pancreatic cancer, colorectal cancer, bladder cancer, osteosarcoma, leiomyosarcoma, ovarian cancer, cervical cancer, colon cancer, head and neck cancer, sarcoma, relapsed/refractory rhabdomyosarcoma, non-rhabdomyosarcoma soft tissue sarcoma, Ewing sarcoma, angiosarcoma, epithelioid hemangioendothelioma, and urothelial cell cancer.

15. The method of claim 14, wherein the breast cancer is selected from the group consisting of metastatic breast cancer, triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor negative breast cancer, ductal carcinoma in situ (OCiS), invasive ductal carcinoma, invasive lobular carcinoma, inflammatory breast cancer, Paget disease of the nipple, phyllodes tumor, and a hormone responsive cancer.

16. The method of claim 12, wherein the cancer is a CDK4/6-replication dependent cancer.

17. The method of claim 12, wherein the cancer is a CDK4/6 replication independent cancer.

18. The method of claim 12, wherein the human is administered the CDK4/6 inhibitor about 30 minutes or less prior to administration of eribulin, or its pharmaceutically acceptable salt.

19. The method of claim 12, wherein the eribulin is administered on days 1 and 8 of a 21-day chemotherapeutic cycle, and the CDK4/6 inhibitor is administered on days 1 and 8 of a 21-day chemotherapeutic cycle.

20. The method of claim 12, wherein the eribulin is administered on days 1, 8, and 15 of a 28-day chemotherapeutic cycle, and the CDK4/6 inhibitor is administered on days 1, 8, and 15 of a 28-day chemotherapeutic cycle.

21. The method of claim 15, further comprising the administration of an anti-hormonal agent, wherein the anti-hormonal agent is selected from the group consisting of a SERM (selective estrogen receptor modulator), a SERD (selective estrogen receptor degrader), a complete estrogen receptor degrader, or another form of partial or complete estrogen antagonist, selective androgen receptor modulator, a selective androgen receptor degrader, a complete androgen receptor degrader, and another form of partial or complete androgen antagonist.

22. The method of claim 21, wherein the anti-hormonal agent is selected from the group consisting of fulvestrant, tamoxifen, anastrozole, letrozole, exemestane, goserelin, leuprolide, megestrol acetate and toremifene.
